(19) United States
(12) Patent Application Publication
(10) Pub. No.: US 2019/0203204 A1
(43) Pub. Date: Jul. 4, 2019

(54) Methods of De Novo Assembly of Barcoded Genomic DNA Fragments

(71) Applicant: President and Fellows of Harvard College, Cambridge, MA (US)

(72) Inventors: Xiaoliang Sunney Xie, Lexington, MA (US); Dong Xing, Cambridge, MA (US); Chi-Han Chang, Cambridge, MA (US)

(21) Appl. No.: 16/324,142

(22) PCT Filed: Aug. 9, 2017
(86) PCT No.: PCT/US17/46060

§ 371 Date: Feb. 8, 2019

Related U.S. Application Data
(60) Provisional application No. 62/373,057, filed on Aug. 10, 2016.

(51) Int. Cl. C12N 15/10

(52) U.S. Cl. CPC C12N 15/1082; C12N 15/1031

(57) ABSTRACT

The present disclosure provides a method for de novo assembly of genomic DNA using barcoded fragments.

### RELATED APPLICATION DATA

This application claims priority to U.S. Provisional Application No. 62/373,057 filed on Aug. 10, 2016 which is hereby incorporated herein by reference in its entirety for all purposes.

### STATEMENT OF GOVERNMENT INTERESTS

This invention was made with government support under 5DP1CA186693 from the National Institutes of Health. The Government has certain rights in the invention.

### BACKGROUND

### Field of the Invention

Embodiments of the present invention relate in general to methods and compositions for the de novo assembly of genomic nucleic acids, such as DNA from a single cell.

### Description of Related Art

De novo genome assembly is the process of assembling individual short sequencing reads into longer sequences without the aid of a reference sequence. Currently, most high throughput sequences generate sequence lengths of only a few hundred base pairs. The short fragments are then reconstructed together by determining where these fragments overlap. However, there are a great number of repetitive sequences in the genome of a complex organism like a human being. Many of those repetitive regions are longer than the read length of a DNA sequencer, which makes it difficult to assemble the whole genome without gaps.

The capability to perform single-cell genome sequencing is important in studies where cell-to-cell variation and population heterogeneity play a key role, such as tumor growth, stem cell reprogramming, embryonic development, etc. Single cell genome sequencing is also important when the cell samples subject to sequencing are precious or rare or in minute amounts. Important to accurate single-cell genome sequencing is the initial amplification of the genomic DNA which can be in minute amounts.

De novo genome assembly after amplification and sequencing is an important aspect of many methods that are used with whole genome sequencing. Whole genome amplification methods include multiple displacement amplification (MDA) which is a common method used in the art with genomic DNA from a single cell prior to sequencing and other analysis. In this method, random primer annealing is followed by extension taking advantage of a DNA polymerase with a strong strand displacement activity. The original genomic DNA from a single cell is amplified exponentially in a cascade-like manner to form hyperbranched DNA structures. Another method of amplifying genomic DNA from a single cell is described in Zong, C., Lu, S., Chapman, A. R., and Xie, X. S. (2012), Genome-wide detection of single-nucleotide and copy-number variations of a single human cell, Science 338, 1622-1626 which describes Multiple Annealing and Looping-Based Amplification Cycles (MALBAC). Another method known in the art is degenerate oligonucleotide primed PCR or DOP-PCR. Several other methods used with single cell genomic DNA include Cheung, V. G. and S. F. Nelson, Whole genome amplification using a degenerate oligonucleotide primer allows hundreds of genotypes to be performed on less than one nanogram of genomic DNA, Proceedings of the National Academy of Sciences of the United States of America, 1996. 93(25): p. 14676-9; Telenius, H., et al., Degenerate oligonucleotide-primed PCR: general amplification of target DNA by a single degenerate primer, Genomics, 1992. 13(3): p. 718-25; Zhang, L., et al., Whole genome amplification from a single cell: implications for genetic analysis. Proceedings of the National Academy of Sciences of the United States of America, 1992, 89(13): p. 5847-51; Lao, K., N. L. Xu, and N. A. Straus, Whole genome amplification using single-primer PCR, Biotechnology Journal, 2008, 3(3): p. 378-82; Dean, F. B., et al., Comprehensive human genome amplification using multiple displacement amplification, Proceedings of the National Academy of Sciences of the United States of America, 2002. 99(8): p. 5261-6; Lage, J. M., et al., Whole genome analysis of genetic alterations in small DNA samples using hyperbranched strand displacement amplification and array-CGH, Genome Research, 2003, 13(2): p. 294-307; Spits, C., et al., Optimization and evaluation of single-cell whole-genome multiple displacement amplification, Human Mutation, 2006, 27(5): p. 496-503; Gole, J., et al., Massively parallel polymerase cloning and genome sequencing of single cells using nanoliter microwells, Nature Biotechnology, 2013. 31(12): p. 1126-32; Jiang, Z., et al., Genome amplification of single sperm using multiple displacement amplification, Nucleic Acids Research, 2005, 33(10): p. e91; Wang, J., et al., Genome-wide Single-Cell Analysis of Recombination Activity and De Novo Mutation Rates in Human Sperm, Cell, 2012. 150(2): p. 402-12; Ni, X., Reproducible copy number variation patterns among single circulating tumor cells of lung cancer patients, PNAS, 2013, 110, 21082-21088; Navin, N., Tumor evolution inferred by single cell sequencing, Nature, 2011, 472 (7341):90-94; Evrony, G. D., et al., Single-neuron sequencing analysis of 11 retrotransposition and somatic mutation in the human brain, Cell, 2012. 151(3): p. 483-96; and McLean, J. S., et al., Genome of the pathogen Porphyromonas gingivalis recovered from a biofilm in a hospital sink using a high-throughput single-cell genomics platform, Genome Research, 2013. 23(5): p. 867-77. Methods directed to aspects of whole genome amplification are reported in WO 2012/166425, U.S. Pat. No. 7,718,403, US 2003/0108870 and U.S. Pat. No. 7,402,386.

However, a need exists for further methods of amplifying small amounts of genomic DNA, such as from a single cell or a small group of cells where the amplicons can be de novo assembled into the genomic DNA.

### SUMMARY

The present disclosure provides a method for genomic DNA fragmentation where adjoining ends of fragments are barcoded with the same unique end barcode sequence during the fragmentation process such that the sequenced fragments can be later computationally assembled into larger sequences by linking the fragments having the same unique end barcode sequences. According to one aspect, a transposome library is used to make fragments of genomic DNA in aqueous media where a unique barcode sequence is inserted or attached to each end of the genomic DNA at a site which has been cut by the transposase of the transposome. The present disclosure contemplates fragmenting genomic DNA into a plurality of fragments, such as 5 or more fragments, 10 or more fragments, 100 or more fragments, 1000 or more fragments, 10,000 or more fragments, 100,000 or more fragments, 1,000,000 or more fragments, or 10,000,000 or more fragments using a transposome library as described herein. According to one aspect, a transposome library includes 5 to 10 transposome members, 10 to 100 transposome members, 100 or more transposome members, 1000 or more transposome members, 10,000 or more transposome members, 100,000 or more transposome members, 1,000,000 or more transposome members, or 10,000,00 or more transposome members. According to one aspect, each transposome includes two transposases and two transposon DNA. The transposon DNA includes a transposase binding site, a barcode and a primer binding site. According to one aspect, the transposon DNA includes a single transposase binding site, a barcode and a primer binding site. Each transposon DNA is a separate nucleic acid bound to a transposase at the transposase binding site. The transposome is a dimer of two separate transposases each bound to its own transposon DNA. According to one aspect, the transposome includes two separate and individual transposon DNA, each bound to its own corresponding transposase. According to one aspect, the transposome includes only two transposases and only two transposon DNA. According to one aspect, the two transposon DNA as part of the transposome are separate, individual or non-linked transposon DNA, each bound to its own corresponding transposase. As an example, separate and individual transposon DNA as described herein having a single transposon binding site, a barcode and a primer binding site allow for the making of millions of transposomes using a microdroplet approach as the transposome can be assembled by its individual parts of a transposase binding to a corresponding transposon DNA and with two transposases dimerizing to form a transposome and with the two transposon DNA of the transposome having the same barcode sequence.

According to one aspect, each transposome member of the library includes a unique barcode of the same sequence on each transposon DNA of the transposome. In this manner, each transposome includes a pair of unique barcode sequences that are different from the barcode sequence of any other transposome in the transposome library. According to one aspect, the transposome library may include transposome members that have the same barcode, although the number of members having the same barcode is relatively small or insignificant. In this manner, the transposome library may be considered to be a subset of the prepared collection of transposomes, where the subset includes only transposomes with a unique barcode sequence, as the objective is to fragment genomic DNA where each fragment cut site is represented by a unique barcode sequence. It is to be understood that an insignificant number of cut sites may share the same barcode sequence due to transposome library preparation. For example, for a given library preparation method, it is mathematically possible that multiple molecules of transposome with the same barcode pair exist, but the library is prepared such that the number of different barcode sequences significantly exceeds the number of transposome molecules that will actually be inserted into the target genome. For example, for a single human cell whole genome which is 6,000,000,000 base pairs long, 1,000,000 transposomes need to be inserted into the whole genome to get an average fragment length of 6,000 bp. To reach this 6000 bp insertion density, at least 3,000,000,000 molecules of transposome are added into the reaction mixture. For a 14 bp randomly synthesized barcode, there are 4̂14=268,435,456 different barcode sequences, which means for each specific barcode there are 3,000,000,000/268,435,456=11.2 copies of molecules. But no matter how many copies of molecules share the same barcode sequence, the chance of having two molecules of transposome with the same barcode sequence inserted into the genome to create fragments is 1,000,000/268,435,456=0.0037. Using this example, on average, 268 fragments may be linked by barcodes before encountering two different genomic DNA fragments having the same barcode tag or sequence. Methods exist to ensure that each barcode sequence in a transposome library is unique, i.e. beginning with more than 3,000,000,000 barcode sequences.

According to one aspect, for genomes of other sizes, the number of barcodes to be used can be scaled accordingly and is determined by the total number of base pairs in the genome divided by the desired fragment size. For example, for a small genome such as that of a lambda phage, having around 50,000 base pairs, only 9 barcodes are needed for insertion into the genome if having an average fragment length of 6,000 bp, so only 9 transposomes each with its uniquely associated barcode are needed for insertion into the genome. According to one aspect, the average fragment length can also be tuned to be smaller or larger by using more or fewer number of transposomes, which can be accomplished by using more or less concentrated transposome solution, respectively; when the targeted average fragment length is smaller so that the number of total fragments is expectedly larger, the number or barcodes to be used may be tuned to be larger to achieve unique barcoding, and vice versa.

Therefore, according to one aspect, substantially all of the cut sites are represented by a unique barcode sequence, and accordingly, substantially all of the fragments may be de novo assembled. According to one aspect, more than 90% of the cut sites are represented by a unique barcode sequence, more than 95% of the cut sites are represented by a unique barcode sequence, 96% of the cut sites are represented by a unique barcode sequence, 97% of the cut sites are represented by a unique barcode sequence, 98% of the cut sites are represented by a unique barcode sequence, 99% of the cut sites are represented by a unique barcode sequence, 99.5% of the cut sites are represented by a unique barcode sequence, or 100% of the cut sites are represented by a unique barcode sequence.

The transposome library is then used to cut the genomic DNA and each transposome inserts or attaches its barcode sequence, such as a unique barcode sequence, in the transposon DNA at both ends of the cut site. In this manner, adjoining ends of a cut site may be later identified by matching barcode sequences and the adjoining ends may be computationally joined together. According to one aspect, fragments produced by the transposome library have one member of a barcode sequence pair, such as a unique barcode sequence pair, on each end of the fragment. According to one aspect, fragments produced by the transposome library each have one member of a barcode sequence pair, such as a unique barcode sequence pair, on each end of the fragment. After the fragments are amplified and sequenced, the ends of fragments can be computationally linked together by matching barcodes so as to de novo assemble the genomic DNA. Accordingly, methods are provided for the linking of nucleic acid fragments by matching barcode sequences which have been attached to the fragments using a transposase.

According to one aspect, the transposon DNA of the transposome can include sequences facilitating amplification methods, such as specific primer sequences or transcription sequences which can be attached to the fragments so that the fragments can be amplified prior to sequencing, such as by PCR or RNA transcription using methods known to those of skill in the art. It is to be understood that the present disclosure contemplates different amplification methods for amplifying the fragments and different sequencing methods for sequencing the amplicons and the methods for de novo genome assembly are not limited to any particular amplification or sequencing method.

Embodiments of the present disclosure are directed to a method of de novo assembly of DNA such as a small amount of genomic DNA or a limited amount of DNA such as a genomic sequence or genomic sequences obtained from a single cell or a plurality of cells of the same cell type or from a tissue, fluid or blood sample obtained from an individual or a substrate. According to certain aspects of the present disclosure, the methods described herein can be performed in a single tube with a single reaction mixture. According to certain aspects of the present disclosure, the nucleic acid sample can be within an unpurified or unprocessed lysate from a single cell. Nucleic acids to be subjected to the methods disclosed herein need not be purified, such as by column purification, prior to being contacted with the various reagents and under the various conditions as described herein. The barcode methods described herein aid in the de novo assembly of fragmented DNA so as to assist in providing substantial and uniform coverage of the entire genome of a single cell producing amplified DNA for high-throughput sequencing.

Embodiments of the present invention relate in general to methods and compositions for making DNA fragments, for example, DNA fragments from the whole genome of a single cell which may then be subjected to amplification and sequencing methods known to those of skill in the art and as described herein. According to certain aspects, methods of making nucleic acid fragments described herein utilize a transposome library. According to one aspect, a transposase as part of a transposome is used to create a set of double stranded genomic DNA fragments. According to certain aspects, the transposases have the capability to bind to transposon DNA and dimerize when contacted together, such as when being placed within a reaction vessel or reaction volume, forming a transposase/transposon DNA complex dimer called a transposome. Each transposon DNA of the transposome includes a double stranded transposase binding site and a first nucleic acid sequence including a barcode sequence unique to the transposome and an amplification promoting sequence, such as a specific priming site (“primer binding site”) or a transcription promoter site. The first nucleic acid sequence may be in the form of a single stranded extension. Each transposome of the transposome library includes a unique barcode sequence that is different from the barcode sequence of each remaining member of the transposome library.

The transposomes have the capability to randomly bind to target locations along double stranded nucleic acids, such as double stranded genomic DNA, forming a complex including the transposome and the double stranded genomic DNA. The transposases in the transposome cleave the double stranded genomic DNA, with one transposase cleaving the upper strand and one transposase cleaving the lower strand. Each of the transposon DNA in the transposome is attached to the double stranded genomic DNA at each end of the cut site, i.e. one transposon DNA of the transposome is attached to the left hand cut site and the other transposon DNA of the transposome is attached to the right hand cut site. In this manner, the left hand cut site and the right hand cut site are barcoded with the same barcode sequence which is unique to the cut site. Accordingly, the barcode sequence identifies the left hand cut site and the right had cut site as being directly adjoining to each other for de novo genome assembly.

According to certain aspects, a plurality of transposase/transposon DNA complex dimers, i.e. transposomes, bind to a corresponding plurality of target locations along a double stranded genomic DNA, for example, and then cleave the double stranded genomic DNA into a plurality of double stranded fragments with each fragment having transposon DNA with a different barcode sequence attached at each end of the double stranded fragment. In this manner and consistent with the above description, each fragment can be computationally placed in sequence by identifying corresponding ends of fragments having the same barcode sequence and computationally linking the ends of the fragments together.

According to one aspect, the transposon DNA is attached to the double stranded genomic DNA and a single stranded gap exists between one strand of the genomic DNA and one strand of the transposon DNA. According to one aspect, gap extension is carried out to fill the gap and create a double stranded connection between the double stranded genomic DNA and the double stranded transposon DNA. According to one aspect, a nucleic acid sequence including the transposase binding site, the barcode sequence and the amplification promoting sequence of the transposon DNA is attached at each end of the double stranded fragment. According to certain aspects, the transposase is attached to the transposon DNA which is attached at each end of the double stranded fragment. According to one aspect, the transposases are removed from the transposon DNA which is attached at each end of the double stranded genomic DNA fragments.

According to one aspect of the present disclosure, the double stranded genomic DNA fragments produced by the transposases which have the transposon DNA with different barcode sequences attached at each end of the double stranded genomic DNA fragments are then gap filled and extended using the transposon DNA as a template. Accordingly, a double stranded nucleic acid extension product is produced which includes the double stranded genomic DNA fragment and a double stranded transposon DNA including a different barcode sequence and an amplification promoting sequence at each end of the double stranded genomic DNA.

At this stage, the double stranded nucleic acid extension products including the genomic DNA fragment, the different barcodes at each end and the amplification promoting sequence can be amplified using methods known to those of skill in the art to produce amplicons of the genomic DNA fragment and the different barcodes at each end. The amplification promoting sequence can be a specific primer binding site at each end of the double stranded genomic DNA. The reference to a “specific” primer binding site indicates that the two primer binding sites have the same sequence and so a primer of a common sequence can be used for amplification of all fragments. PCR primer sequences and reagents can be used for amplification. The amplification promoting sequence can be an RNA polymerase binding site for production of RNA transcripts which may then be reverse transcribed into cDNA for linear amplification. The double stranded nucleic acid extension products including the genomic DNA fragment, the different barcodes at each end and the amplification promoting sequence can be combined with amplification reagents and the double stranded genomic nucleic acid fragment may then be amplified using methods known to those of skill in the art to produce amplicons of the double stranded genomic nucleic acid fragment.

The amplicons can then be collected and/or purified prior to further analysis. The amplicons can be sequenced using methods known to those of skill in the art. Once sequenced, the sequences can be computationally analyzed to identify fragment ends having the same barcode sequence and the fragment ends can be computationally joined to one another to create longer sequences for de novo assembly of the genomic DNA. In one embodiment, when the genomic DNA is from a single cell with more than one ploidy, de novo assembly of the genome can achieve a haplotype-resolved de novo assembly, when unique barcode sequences are inserted into each fragment end of each fragment of two alleles.

Embodiments of the present disclosure are directed to a method of amplifying DNA using a barcoded fragments as described herein, wherein the DNA is a small amount of genomic DNA or a limited amount of DNA such as a genomic sequence or genomic sequences obtained from a single cell or a plurality of cells of the same cell type or from a tissue, fluid or blood sample obtained from an individual or a substrate. According to certain aspects of the present disclosure, the methods described herein can be performed in a single tube to create the barcoded fragments which are then amplified and sequenced using high throughput sequencing platforms known to those of skill in the art and then computationally joined end to end, using methods and software known to those of skill in the art, by matching barcode sequences which designate cut or fragmentation sites between adjoining fragments of the original nucleic acid sequence.

The transposome fragmentation and barcoding method described herein is useful for amplifying, sequencing and de novo assembling of small or limited amounts of DNA. Methods described herein have particular application in biological systems or tissue samples characterized by highly heterogeneous cell populations such as tumor and neural masses. Methods described herein to amplify and sequence barcoded genomic DNA fragments facilitate the analysis and de novo assembly of such amplified DNA using next generation sequencing techniques known to those of skill in the art and described herein. The methods described herein can utilize varied sources of DNA materials, including genetically heterogeneous tissues (e.g. cancers), rare and precious samples (e.g. embryonic stem cells), and non-dividing cells (e.g. neurons) and the like, as well as, sequencing platforms and genotyping methods known to those of skill in the art.

Further features and advantages of certain embodiments of the present disclosure will become more fully apparent in the following description of the embodiments and drawings thereof, and from the claims.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1 depicts in schematic a structure of a transposon DNA with a 5′ extension being linear, where T is the double stranded transposase binding site, P is a priming site at one end of the extension and B is a barcode sequence.

FIG. 2 is a schematic of a general embodiment of transposase and transposon DNA spontaneously forming a transposome, which may occur within a droplet or other formation media.

FIG. 3 is a schematic of transposome binding to genomic DNA, cutting into fragments and addition or insertion of transposon DNA including a primer binding site (purple), a transposase binding site (light blue) and a unique barcode sequence represented in each transposome by different colors.

FIG. 4 is a schematic of transposase removal, gap filling and extension to form nucleic acid extension products including genomic DNA, primer binding site, barcode sequence and transposase binding site.

FIG. 5 is a schematic of the use of barcodes to chain short sequencing reads into a longer continuous sequence.

FIG. 6 depicts a microparticle or bead having a plurality of transposon DNA attached thereto by a linker and having a cleavage site for cleavage of the transposon DNA from the microparticle or bead.

FIG. 7 is a schematic of using microdroplets to isolate microparticles containing transposon DNA with specific barcodes and the creation of transposomes having the same barcode pair within each microdroplet.

FIG. 8 is a schematic of microfluidic circuits for use in preparing barcoded transposomes.

FIG. 9 is a schematic of insertion of transposomes carrying different pairs of barcodes to two alleles of a diploid genome and haplotyping of the genome.

### DETAILED DESCRIPTION

The practice of certain embodiments or features of certain embodiments may employ, unless otherwise indicated, conventional techniques of molecular biology, microbiology, recombinant DNA, and so forth which are within ordinary skill in the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), ANIMAL CELL CULTURE (R. I. Freshney, Ed., 1987), the series METHODS IN ENZYMOLOGY (Academic Press, Inc.); GENE TRANSFER VECTORS FOR MAMMALIAN CELLS (J. M. Miller and M. P. Calos eds. 1987), HANDBOOK OF EXPERIMENTAL IMMUNOLOGY, (D. M. Weir and C. C. Blackwell, Eds.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987), CURRENT PROTOCOLS IN IMMUNOLOGY (J. E. coligan, A. M. Kruisbeek, D. H. Margulies, E. M. Shevach and W. Strober, eds., 1991); ANNUAL REVIEW OF IMMUNOLOGY; as well as monographs in journals such as ADVANCES IN IMMUNOLOGY. All patents, patent applications, and publications mentioned herein, both supra and infra, are hereby incorporated herein by reference.

Terms and symbols of nucleic acid chemistry, biochemistry, genetics, and molecular biology used herein follow those of standard treatises and texts in the field, e.g., Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

The present invention is based in part on the discovery of methods for making nucleic acid fragment templates, such as from DNA or genomic DNA, using a transposase or transposome to fragment the original or starting nucleic acid sequence, such as genomic DNA, and to attach a barcode sequence to each end of a cut or fragmentation site to facilitate the later computational rejoining of fragment sequences as part of a de novo assembly process. The method described herein may be referred to as “chaine annotation via transposon insertion” or “CHIANTI.” The barcoded nucleic acid fragment templates are amplified to produce amplicons. The amplicons of the nucleic acid fragment templates may be collected and sequenced. The collected amplicons form a library of amplicons of the fragments of the original nucleic acid, such as genomic DNA.

According to one aspect, a genomic DNA, such as genomic nucleic acid obtained from a lysed single cell, is obtained. A plurality or library of transposomes is used to cut the genomic DNA into double stranded fragments. Each transposome of the plurality or library is a dimer of a transposase bound to a transposon DNA, i.e. each transposome includes two separate transposon DNA. Each transposon DNA of a transposome includes a transposase binding site, a barcode sequence unique to the transposome and an amplification facilitating sequence, such as a specific primer binding site.

The barcode sequence of each transposon DNA of a transposome is the same sequence and is unique to the transposome. Each transposome of the plurality or library of transposomes has its own unique representative barcode sequence which is different from the remaining members of the transposome plurality or library. The transposon DNA becomes attached to the upper and lower strands of each double stranded fragment at each cut or fragmentation site. Since the barcode sequence is the same for each transposon DNA, the cut or fragmentation site is tagged with the same barcode sequence which can be later identified to computationally rejoin the cut or fragmentation site. Since each transposome has its own unique barcode sequence, and a library of transposomes are used to create many cut or fragmentation sites, each cut or fragmentation site will have its own unique barcode sequence. Accordingly, many fragments from the original nucleic acid sequence are created by the library of transposomes with each fragment having a dissimilar barcode at each end of the fragment. The double stranded fragments are then processed to fill gaps. The fragments are amplified using suitable amplification reagents, such as a specific primer sequence, DNA polymerase and nucleotides for PCR amplification and are sequenced using methods known to those of skill in the art. Matching barcodes are identified which indicate cut or fragmentation sites and the matching barcodes are used to computationally rejoin fragments to recreate the original nucleic acid sequence.

DNA fragment templates made using the transposase methods described herein can be amplified within microdroplets using methods known to those of skill in the art. Microdroplets may be formed as an emulsion of an oil phase and an aqueous phase. An emulsion may include aqueous droplets or isolated aqueous volumes within a continuous oil phase Emulsion whole genome amplification methods are described using small volume aqueous droplets in oil to isolate each fragment for uniform amplification of a single cell's genome. By distributing each fragment into its own droplet or isolated aqueous reaction volume, each droplet is allowed to reach saturation of DNA amplification. The amplicons within each droplet are then merged by demulsification resulting in an even amplification of all of the fragments of the whole genome of the single cell.

In certain aspects, amplification is achieved using PCR. PCR is a reaction in which replicate copies are made of a target polynucleotide using a pair of primers or a set of primers consisting of an upstream and a downstream primer, and a catalyst of polymerization, such as a DNA polymerase, and typically a thermally-stable polymerase enzyme. Methods for PCR are well known in the art, and taught, for example in MacPherson et al. (1991) PCR 1: A Practical Approach (IRL Press at Oxford University Press). The term “polymerase chain reaction” (“PCR”) of Mullis (U.S. Pat. Nos. 4,683,195, 4,683,202, and 4,965,188) refers to a method for increasing the concentration of a segment of a target sequence without cloning or purification. This process for amplifying the target sequence includes providing oligonucleotide primers with the desired target sequence and amplification reagents, followed by a precise sequence of thermal cycling in the presence of a polymerase (e.g., DNA polymerase). The primers are complementary to their respective strands (“primer binding sequences”) of the double stranded target sequence. To effect amplification, the double stranded target sequence is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle;” there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”) and the target sequence is said to be “PCR amplified.” The PCR amplification reaches saturation when the double stranded DNA amplification product accumulates to a certain amount that the activity of DNA polymerase is inhibited. Once saturated, the PCR amplification reaches a plateau where the amplification product does not increase with more PCR cycles.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself within each microdroplet are, themselves, efficient templates for subsequent PCR amplifications. Methods and kits for performing PCR are well known in the art. All processes of producing replicate copies of a polynucleotide, such as PCR or gene cloning, are collectively referred to herein as replication. A primer can also be used as a probe in hybridization reactions, such as Southern or Northern blot analyses.

The expression “amplification” or “amplifying” refers to a process by which extra or multiple copies of a particular polynucleotide are formed. Amplification includes methods such as PCR, ligation amplification (or ligase chain reaction, LCR) and other amplification methods. These methods are known and widely practiced in the art. See, e.g., U.S. Pat. Nos. 4,683,195 and 4,683,202 and Innis et al., “PCR protocols: a guide to method and applications” Academic Press, Incorporated (1990) (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting amplification reactions are commercially available. Primers useful to amplify sequences from a particular gene region are preferably complementary to, and hybridize specifically to sequences in the target region or in its flanking regions and can be prepared using methods known to those of skill in the art. Nucleic acid sequences generated by amplification can be sequenced directly.

When hybridization occurs in an antiparallel configuration between two single-stranded polynucleotides, the reaction is called “annealing” and those polynucleotides are described as “complementary”. A double-stranded polynucleotide can be complementary or homologous to another polynucleotide, if hybridization can occur between one of the strands of the first polynucleotide and the second. Complementarity or homology (the degree that one polynucleotide is complementary with another) is quantifiable in terms of the proportion of bases in opposing strands that are expected to form hydrogen bonding with each other, according to generally accepted base-pairing rules.

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences. According to one aspect of the present disclosure, each microdroplet includes PCR product of a single template DNA fragment.

The term “amplification reagents” may refer to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.). Amplification methods include PCR methods known to those of skill in the art and also include rolling circle amplification (Blanco et al., J. Biol. Chem., 264, 8935-8940, 1989), hyperbranched rolling circle amplification (Lizard et al., Nat. Genetics, 19, 225-232, 1998), and loop-mediated isothermal amplification (Notomi et al., Nuc. Acids Res., 28, e63, 2000) each of which are hereby incorporated by reference in their entireties.

For emulsion PCR, an emulsion PCR reaction is created by vigorously shaking or stirring a “water in oil” mix to generate millions of micron-sized aqueous compartments. Microfluidic chips may be equipped with a device to create an emulsion by shaking or stirring an oil phase and a water phase. Alternatively, aqueous droplets may be spontaneously formed by combining a certain oil with an aqueous phase or introducing an aqueous phase into an oil phase. The DNA library to be amplified is mixed in a limiting dilution prior to emulsification. The combination of compartment size, i.e. microdroplet size, and amount of microdroplets created limiting dilution of the DNA fragment library to be amplified is used to generate compartments containing, on average, just one DNA molecule. Depending on the size of the aqueous compartments generated during the microdroplet formation or emulsification step, up to 3×10⁹ individual PCR reactions per μl can be conducted simultaneously in the same tube. Essentially each little aqueous compartment microdroplet in the emulsion forms a micro PCR reactor. The average size of a compartment in an emulsion ranges from sub-micron in diameter to over a 100 microns, or from 1 picoliter to 1000 picoliters or from 1 nanoliter to 1000 nanoliters or from 1 picoliter to 1 nanoliter or from 1 picoliter to 1000 nanoliters depending on the emulsification conditions.

Other amplification methods, as described in British Patent Application No. GB 2,202,328, and in PCT Patent Application No. PCT/US89/01025, each incorporated herein by reference, may be used in accordance with the present disclosure. In the former application, “modified” primers are used in a PCR-like template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other suitable amplification methods include “race and “one-sided PCR.”. (Frohman, In: PCR Protocols: A Guide To Methods And Applications, Academic Press, N.Y., 1990, each herein incorporated by reference). Methods based on ligation of two (or more) oligonucleotides in the presence of nucleic acid having the sequence of the resulting “di-oligonucleotide,” thereby amplifying the di-oligonucleotide, also may be used to amplify DNA in accordance with the present disclosure (Wu et al., Genomics 4:560-569, 1989, incorporated herein by reference).

According to certain aspects, an exemplary transposon system includes Tn5 transposase, Mu transposase, Tn7 transposase or IS5 transposase and the like. Other useful transposon systems are known to those of skill in the art and include Tn3 transposon system (see Maekawa, T., Yanagihara, K., and Ohtsubo, E. (1996), A cell-free system of Tn3 transposition and transposition immunity, Genes Cells 1, 1007-1016), Tn7 transposon system (see Craig, N. L. (1991), Tn7: a target site-specific transposon, Mol. Microbiol. 5, 2569-2573), Tn10 tranposon system (see Chalmers, R., Sewitz, S., Lipkow, K., and Crellin, P. (2000), Complete nucleotide sequence of Tn10, J. Bacteriol 182, 2970-2972), Piggybac transposon system (see Li, X., Burnight, E. R., Cooney, A. L., Malani, N., Brady, T., Sander, J. D., Staber, J., Wheelan, S. J., Joung, J. K., McCray, P. B., Jr., et al. (2013), PiggyBac transposase tools for genome engineering, Proc. Natl. Acad. Sci. USA 110, E2279-2287), Sleeping beauty transposon system (see Ivics, Z., Hackett, P. B., Plasterk, R. H., and Izsvak, Z. (1997), Molecular reconstruction of Sleeping Beauty, a Tc1-like transposon from fish, and its transposition in human cells, Cell 91, 501-510), Tol2 transposon system (see Kawakami, K. (2007), Tol2: a versatile gene transfer vector in vertebrates, Genome Biol. 8 Suppl. 1, S7.)

DNA to be amplified may be obtained from a single cell or a small population of cells. Methods described herein allow DNA to be amplified from any species or organism in a reaction mixture, such as a single reaction mixture carried out in a single reaction vessel. In one aspect, methods described herein include sequence independent amplification of DNA from any source including but not limited to human, animal, plant, yeast, viral, eukaryotic and prokaryotic DNA.

According to one aspect, a method of single cell whole genome amplification, sequencing and de novo assembly is provided which includes contacting double stranded genomic DNA from a single cell with Tn5 transposases each bound to a transposon DNA, wherein the transposon DNA includes a double-stranded 19 bp transposase (Tnp) binding site and a first nucleic acid sequence including one or more of a barcode sequence and a primer binding site to form a transposase/transposon DNA complex dimer called a transposome. The first nucleic acid sequence may be in the form of a single stranded extension. According to one aspect, the first nucleic acid sequence may be an overhang, such as a 5′ overhang, wherein the overhang includes a barcode region and a priming site. The overhang can be of any length suitable to include a barcode region and a priming site as desired. The transposome bind to target locations along the double stranded genomic DNA and cleave the double stranded genomic DNA into a plurality of double stranded fragments, with each double stranded fragment having a first complex attached to an upper strand by the Tnp binding site and a second complex attached to a lower strand by the Tnp binding site. The transposon binding site, and therefore the transposon DNA, is attached to each 5′ end of the double stranded fragment. According to one aspect, the Tn5 transposases are removed from the complex. The double stranded fragments are extended along the transposon DNA to make a double stranded extension product having dissimilar barcode sequences and specific primer binding sites at each end of the double stranded extension product. According to one aspect, a gap which may result from attachment of the Tn5 transposase binding site to the double stranded genomic DNA fragment may be filled. The gap filled double stranded extension product is mixed with amplification reagents, and the double stranded genomic DNA fragment is amplified. The amplicons, which include a dissimilar barcode sequence at each end, are sequenced using, for example, high-throughput sequencing methods known to those of skill in the art.

In a particular aspect, embodiments are directed to methods for the amplification, sequencing and de novo assembly of substantially the entire genome without loss of representation of specific sites (herein defined as “whole genome amplification”). In a specific embodiment, whole genome amplification comprises amplification of substantially all fragments or all fragments of a genomic library. In a further specific embodiment, “substantially entire” or “substantially all” refers to about 80%, about 85%, about 90%, about 95%, about 97%, or about 99% of all sequences in a genome.

According to one aspect, the DNA sample is genomic DNA, micro dissected chromosome DNA, yeast artificial chromosome (YAC) DNA, plasmid DNA, cosmid DNA, phage DNA, P1 derived artificial chromosome (PAC) DNA, or bacterial artificial chromosome (BAC) DNA, mitochondrial DNA, chloroplast DNA, forensic sample DNA, or other DNA from natural or artificial sources to be tested. In another preferred embodiment, the DNA sample is mammalian DNA, plant DNA, yeast DNA, viral DNA, or prokaryotic DNA. In yet another preferred embodiment, the DNA sample is obtained from a human, bovine, porcine, ovine, equine, rodent, avian, fish, shrimp, plant, yeast, virus, or bacteria. Preferably the DNA sample is genomic DNA.

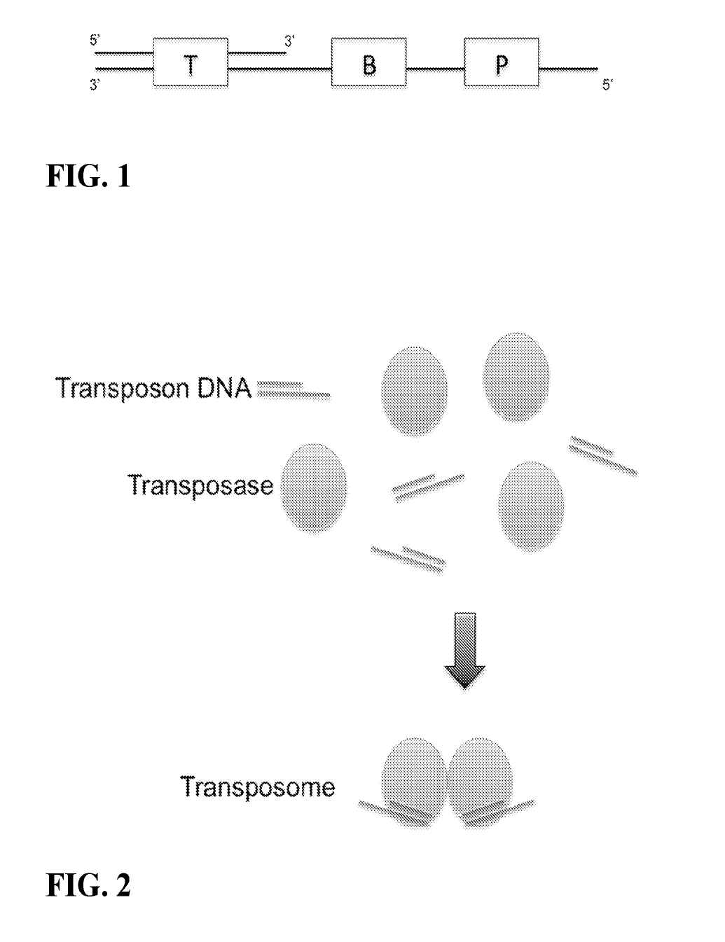

According to certain exemplary aspects, a transposition system is used to make nucleic acid fragments for amplification, sequencing and de novo assembly as desired. According to one aspect, a transposition system is used to fragment genomic DNA into double stranded genomic DNA fragments with the transposon DNA having the same barcode inserted therein. As illustrated in FIG. 1, a transposon DNA includes a double stranded transposase binding site, a barcode sequence B and a priming site P. The double stranded transposase binding site may be a double-stranded 19 bp Tn5 transposase (Tnp) binding site which is linked or connected, such as by covalent bond, to a single-stranded overhang including a barcode region and a priming site at one end of the overhang. The transposon DNA is inserted into the genomic DNA of a single cell while creating millions of small fragments using a transposase. After transposase removal and gap fill-in, the genomic DNA fragments having dissimilar barcode sequences and a specific primer sequence at each end of the fragment are amplified using specific primers together with a DNA polymerase, nucleotides and amplification reagents to PCR amplify the whole genome of the single cell.

According to certain aspects when amplifying small amounts of DNA such as DNA from a single cell, a DNA column purification step is not carried out so as to maximize the small amount (-6 pg) of genomic DNA that can be obtained from within a single cell prior to amplification. The DNA can be amplified directly from a cell lysate or other impure condition. Accordingly, the DNA sample may be impure, unpurified, or not isolated. Accordingly, aspects of the present method allow one to maximize genomic DNA for amplification and reduce loss due to purification. According to an additional aspect, methods described herein may utilize amplification methods other than PCR.

According to one aspect and as illustrated in general in FIG. 2, transposase (Tnp) and the transposon DNA are combined, such as within a microdroplet and the Tnp and the transposon DNA bind to each other and dimerize to form transposomes.

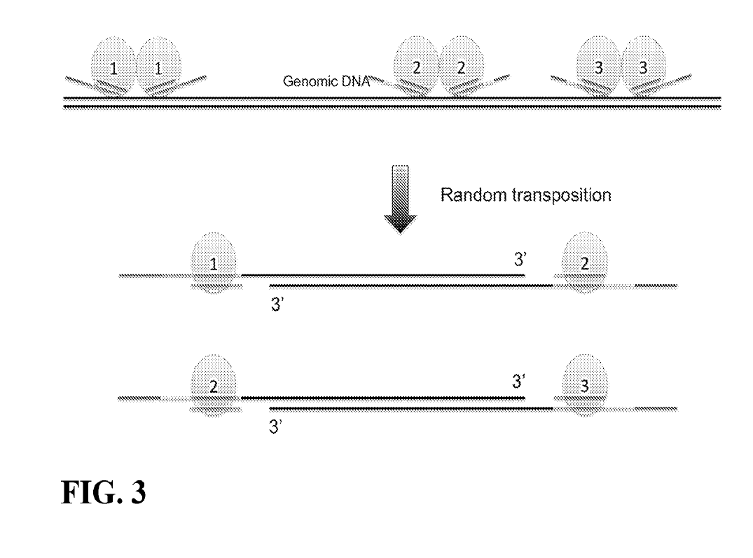

As shown in FIG. 3, the transposomes of the transposome library randomly capture or otherwise bind to the target single-cell genomic DNA as dimers. Representative transposomes are numbered 1, 2 and 3, though the number of transposomes can be in the thousands, ten-thousands, hundred-thousands, millions, etc. Each transposome is represented by a unique barcode sequence, for example barcode sequence 1, barcode sequence 2, barcode sequence 3, etc. The unique barcode sequence is within each transposon DNA of the transposome. Since there are two transposon DNAs per transposome, the two transposon DNAs can be considered a homo dimer, which means one transposon DNA dimer carries two DNA sequences with the same barcode information. Each transposome (and transposon DNA dimer) of the transposome library has a different barcode unique to the transposome. The transposases in the transposome cut the genomic DNA with one transposase cutting an upper strand and one transposase cutting a lower strand to create a genomic DNA fragment. The plurality of transposomes creates a plurality of genomic DNA fragments. One transposon DNA from the transposon DNA dimer is thus attached to each end of the cut site or fragmentation site, i.e., one transposon DNA from transposome 1 is attached to the left hand cut site and the other transposon DNA from transposome 1 is attached to the right hand cut site. Since the transposome library cuts the nucleic acid into fragments, each fragment will have a dissimilar barcode sequence at each end of the fragment, i.e. each fragment is produced by two different cut sites cut by two different transposomes of the transposome library including different barcode sequences. This is represented by the two exemplary fragments where the upper fragment has barcode sequence 1 on one end and barcode sequence 2 on the other end. Likewise, the lower fragment has barcode sequence 2 on one end and barcode sequence 3 on the other end. As illustrated, the cut site between the two fragments is produced by transposome 2 and the left hand cut site (i.e. viewing the right side of the upper fragment in FIG. 3) includes the one transposon with barcode sequence 2 while the right hand cut site (i.e. viewing the left side of the lower fragment in FIG. 3) includes the other transposon with barcode sequence 2.

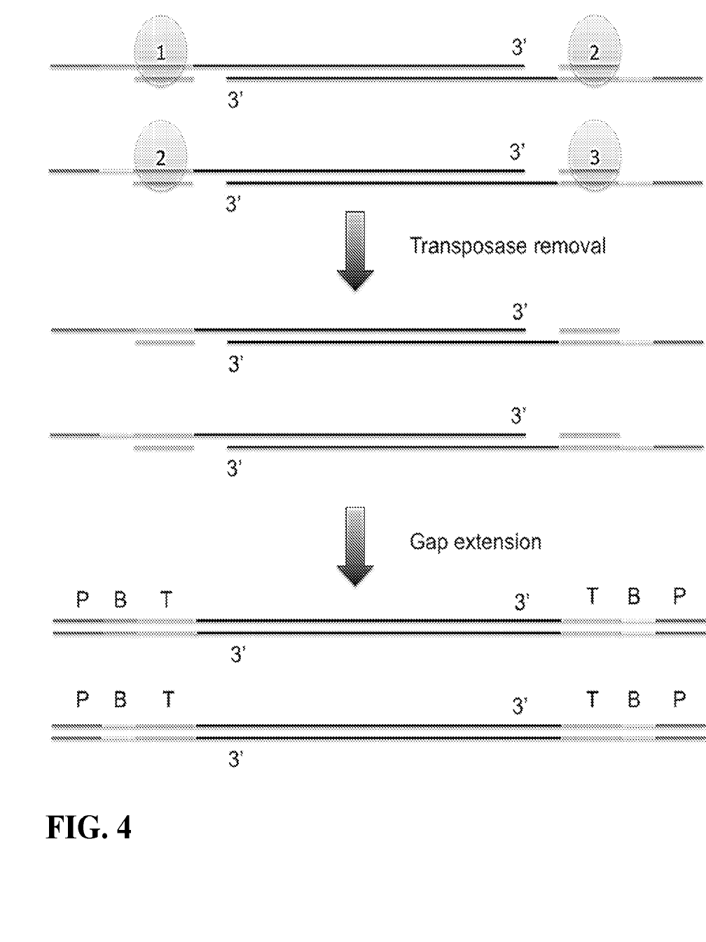

As illustrated in FIG. 4, the fragmentation of the genomic DNA leaves a gap on both ends of the transposition/insertion site. The gap may have any length but a 9 base gap is exemplary. The result is a genomic DNA fragment with a transposon DNA Tnp binding site attached to the 5′ position of an upper strand and a transposon DNA Tnp binding site attached to the 5′ position of a lower strand. Gaps resulting from the attachment or insertion of the transposon DNA are shown. After transposition, the transposase is removed and gap extension is performed to fill the gap and complement the single-stranded overhang originally designed in the transposon DNA as shown in FIG. 4.

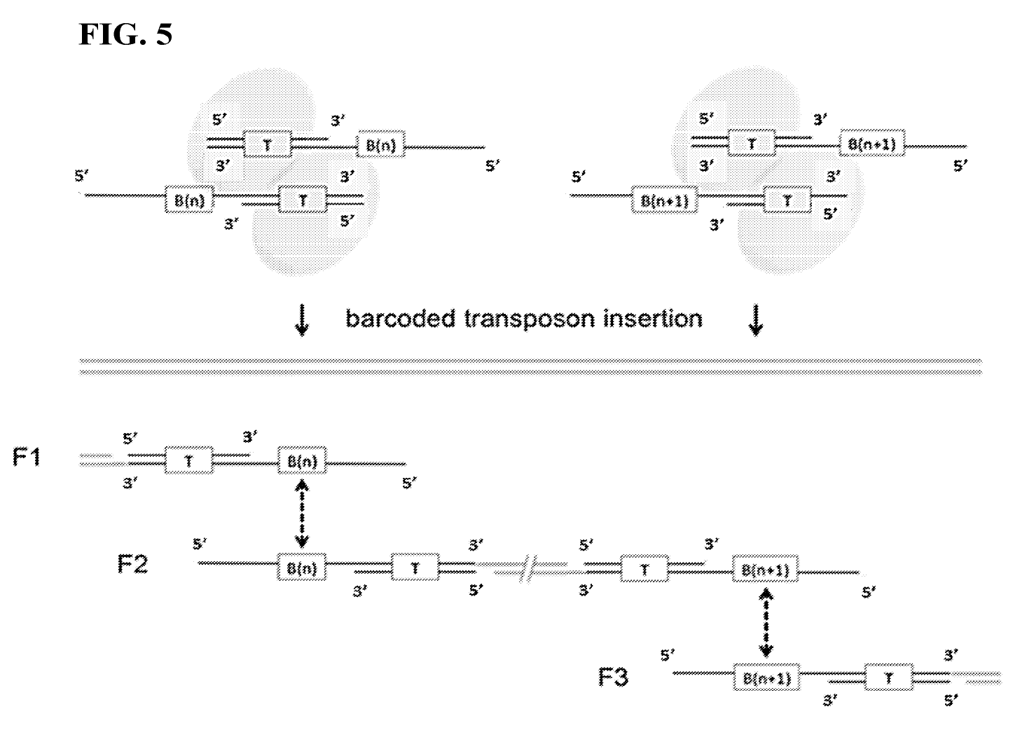

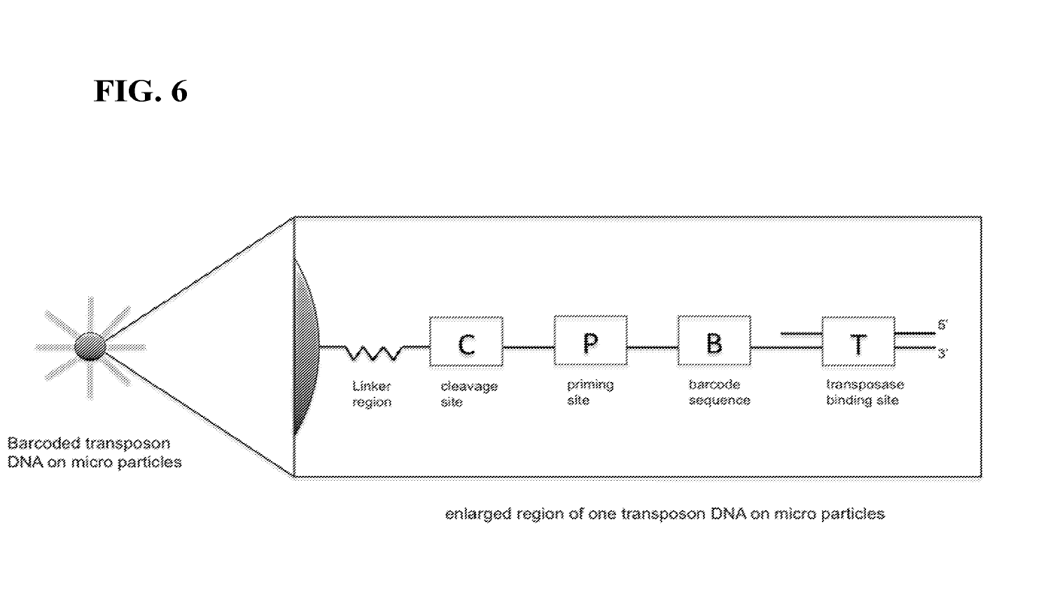

As further illustrated in FIG. 5, a plurality of transposomes n with corresponding barcode sequences Bn are used to create a plurality of fragments and the barcode sequences are used to chain short sequencing reads into longer continuous sequences. A library of transposomes (on the order of millions for example) with each transposome carrying two transposon DNA with the same barcodes B(n) are inserted into the genomic DNA and cut the genomic DNA into millions of different fragments (F1, F2, F3 . . . ). After whole genome amplification and sequencing, the fragments tagged with the same barcodes can be computationally linked together to achieve longer fragment length.

Particular Tn5 transposition systems are described and are available to those of skill in the art. See Goryshin, I. Y. and W. S. Reznikoff, Tn5 in vitro transposition. The Journal of biological chemistry, 1998. 273(13): p. 7367-74; Davies, D. R., et al., Three-dimensional structure of the Tn5 synaptic complex transposition intermediate. Science, 2000. 289(5476): p. 77-85; Goryshin, I. Y., et al., Insertional transposon mutagenesis by electroporation of released Tn5 transposition complexes. Nature biotechnology, 2000. 18(1): p. 97-100 and Steiniger-White, M., I. Rayment, and W. S. Reznikoff, Structure/function insights into Tn5 transposition. Current opinion in structural biology, 2004. 14(1): p. 50-7 each of which are hereby incorporated by reference in their entireties for all purposes. Kits utilizing a Tn5 transposition system for DNA library preparation and other uses are known. See Adey, A., et al., Rapid, low-input, low-bias construction of shotgun fragment libraries by high-density in vitro transposition. Genome biology, 2010. 11(12): p. R119; Marine, R., et al., Evaluation of a transposase protocol for rapid generation of shotgun high-throughput sequencing libraries from nanogram quantities of DNA. Applied and environmental microbiology, 2011. 77(22): p. 8071-9; Parkinson, N. J., et al., Preparation of high-quality next-generation sequencing libraries from picogram quantities of target DNA. Genome research, 2012. 22(1): p. 125-33; Adey, A. and J. Shendure, Ultra-low-input, tagmentation-based whole-genome bisulfite sequencing. Genome research, 2012. 22(6): p. 1139-43; Picelli, S., et al., Full-length RNA-seq from single cells using Smart-seq2. Nature protocols, 2014. 9(1): p. 171-81 and Buenrostro, J. D., et al., Transposition of native chromatin for fast and sensitive epigenomic profiling of open chromatin, DNA-binding proteins and nucleosome position. Nature methods, 2013, each of which is hereby incorporated by reference in its entirety for all purposes. See also WO 98/10077, EP 2527438 and EP 2376517 each of which is hereby incorporated by reference in its entirety. A commercially available transposition kit is marketed under the name NEXTERA and is available from Illumina.

The term “genome” as used herein is defined as the collective gene set carried by an individual, cell, or organelle. The term “genomic DNA” as used herein is defined as DNA material comprising the partial or full collective gene set carried by an individual, cell, or organelle.

As used herein, the term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and 2,2N,N-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide,” “oligonucleotide” and “nucleic acid molecule” are used interchangeably herein and refer to a polymer of nucleotides, either deoxyribonucleotides or ribonucleotides, of any length joined together by a phosphodiester linkage between 5′ and 3′ carbon atoms. Polynucleotides can have any three-dimensional structure and can perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. The term also refers to both double- and single-stranded molecules. Unless otherwise specified or required, any embodiment of this invention that comprises a polynucleotide encompasses both the double-stranded form and each of two complementary single-stranded forms known or predicted to make up the double-stranded form. A polynucleotide is composed of a specific sequence of four nucleotide bases: adenine (A); cytosine (C); guanine (G); thymine (T); and uracil (U) for thymine when the polynucleotide is RNA. Thus, the term polynucleotide sequence is the alphabetical representation of a polynucleotide molecule. This alphabetical representation can be input into databases in a computer having a central processing unit and used for bioinformatics applications such as functional genomics and homology searching.

The terms “DNA,” “DNA molecule” and “deoxyribonucleic acid molecule” refer to a polymer of deoxyribonucleotides. DNA can be synthesized naturally (e.g., by DNA replication). RNA can be post-transcriptionally modified. DNA can also be chemically synthesized. DNA can be single-stranded (i.e., ssDNA) or multi-stranded (e.g., double stranded, i.e., dsDNA).

The terms “nucleotide analog,” “altered nucleotide” and “modified nucleotide” refer to a non-standard nucleotide, including non-naturally occurring ribonucleotides or deoxyribonucleotides. In certain exemplary embodiments, nucleotide analogs are modified at any position so as to alter certain chemical properties of the nucleotide yet retain the ability of the nucleotide analog to perform its intended function. Examples of positions of the nucleotide which may be derivitized include the 5 position, e.g., 5-(2-amino)propyl uridine, 5-bromo uridine, 5-propyne uridine, 5-propenyl uridine, etc.; the 6 position, e.g., 6-(2-amino) propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said analogs in vivo or in vitro.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, the terms “complementary” and “complementarity” are used in reference to nucleotide sequences related by the base-pairing rules. For example, the sequence 5′-AGT-3′ is complementary to the sequence 5′-ACT-3′. Complementarity can be partial or total. Partial complementarity occurs when one or more nucleic acid bases is not matched according to the base pairing rules. Total or complete complementarity between nucleic acids occurs when each and every nucleic acid base is matched with another base under the base pairing rules. The degree of complementarity between nucleic acid strands has significant effects on the efficiency and strength of hybridization between nucleic acid strands.

The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

The term “Tₘ” refers to the melting temperature of a nucleic acid. The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41 (% G+C), when a nucleic acid is in aqueous solution at 1 M NaCl (See, e.g., Anderson and Young, Quantitative Filter Hybridization, in Nucleic Acid Hybridization (1985)). Other references include more sophisticated computations that take structural as well as sequence characteristics into account for the calculation of Tₘ.

The term “stringency” refers to the conditions of temperature, ionic strength, and the presence of other compounds such as organic solvents, under which nucleic acid hybridizations are conducted.

“Low stringency conditions,” when used in reference to nucleic acid hybridization, comprise conditions equivalent to binding or hybridization at 42° C. in a solution consisting of 5×SSPE (43.8 g/l NaCl, 6.9 g/l NaH₂PO₄(H₂O) and 1.85 g/l EDTA, pH adjusted to 7.4 with NaOH), 0.1% SDS, 5× Denhardt's reagent (50× Denhardt's contains per 500 ml: 5 g Ficoll (Type 400, Pharmacia), 5 g BSA (Fraction V; Sigma)) and 100 mg/ml denatured salmon sperm DNA followed by washing in a solution comprising 5×SSPE, 0.1% SDS at 42° C. when a probe of about 500 nucleotides in length is employed.

“Medium stringency conditions,” when used in reference to nucleic acid hybridization, comprise conditions equivalent to binding or hybridization at 42° C. in a solution consisting of 5×SSPE (43.8 g/l NaCl, 6.9 g/l NaH₂PO₄(H₂O) and 1.85 g/l EDTA, pH adjusted to 7.4 with NaOH), 0.5% SDS, 5× Denhardt's reagent and 100 mg/ml denatured salmon sperm DNA followed by washing in a solution comprising 1.0×SSPE, 1.0% SDS at 42° C. when a probe of about 500 nucleotides in length is employed.

“High stringency conditions,” when used in reference to nucleic acid hybridization, comprise conditions equivalent to binding or hybridization at 42° C. in a solution consisting of 5×SSPE (43.8 g/l NaCl, 6.9 g/l NaH₂PO₄(H₂O) and 1.85 g/l EDTA, pH adjusted to 7.4 with NaOH), 0.5% SDS, 5× Denhardt's reagent and 100 mg/ml denatured salmon sperm DNA followed by washing in a solution comprising 0.1×SSPE, 1.0% SDS at 42° C. when a probe of about 500 nucleotides in length is employed.

In certain exemplary embodiments, cells are identified and then a single cell or a plurality of cells is isolated. Cells within the scope of the present disclosure include any type of cell where understanding the DNA content is considered by those of skill in the art to be useful. A cell according to the present disclosure includes a cancer cell of any type, hepatocyte, oocyte, embryo, stem cell, iPS cell, ES cell, neuron, erythrocyte, melanocyte, astrocyte, germ cell, oligodendrocyte, kidney cell and the like. According to one aspect, the methods of the present invention are practiced with the cellular DNA from a single cell. A plurality of cells includes from about 2 to about 1,000,000 cells, about 2 to about 10 cells, about 2 to about 100 cells, about 2 to about 1,000 cells, about 2 to about 10,000 cells, about 2 to about 100,000 cells, about 2 to about 10 cells or about 2 to about 5 cells.

Nucleic acids processed by methods described herein may be DNA and they may be obtained from any useful source, such as, for example, a human sample. In specific embodiments, a double stranded DNA molecule is further defined as comprising a genome, such as, for example, one obtained from a sample from a human. The sample may be any sample from a human, such as blood, serum, plasma, cerebrospinal fluid, cheek scrapings, nipple aspirate, biopsy, semen (which may be referred to as ejaculate), urine, feces, hair follicle, saliva, sweat, immunoprecipitated or physically isolated chromatin, and so forth. In specific embodiments, the sample comprises a single cell. In specific embodiments, the sample includes only a single cell.

In particular embodiments, the amplified and de novo assembled nucleic acid molecule from the sample provides diagnostic or prognostic information. For example, the prepared nucleic acid molecule from the sample may provide genomic copy number and/or sequence information, allelic variation information, cancer diagnosis, prenatal diagnosis, paternity information, disease diagnosis, detection, monitoring, and/or treatment information, sequence information, and so forth.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. Furthermore, in general, cells from any population can be used in the methods, such as a population of prokaryotic or eukaryotic single celled organisms including bacteria or yeast. A single cell suspension can be obtained using standard methods known in the art including, for example, enzymatically using trypsin or papain to digest proteins connecting cells in tissue samples or releasing adherent cells in culture, or mechanically separating cells in a sample. Single cells can be placed in any suitable reaction vessel in which single cells can be treated individually. For example a 96-well plate, such that each single cell is placed in a single well.

Methods for manipulating single cells are known in the art and include fluorescence activated cell sorting (FACS), flow cytometry (Herzenberg., PNAS USA 76:1453-55 1979), micromanipulation and the use of semi-automated cell pickers (e.g. the Quixell™ cell transfer system from Stoelting Co.). Individual cells can, for example, be individually selected based on features detectable by microscopic observation, such as location, morphology, or reporter gene expression. Additionally, a combination of gradient centrifugation and flow cytometry can also be used to increase isolation or sorting efficiency.

Once a desired cell has been identified, the cell is lysed to release cellular contents including DNA, using methods known to those of skill in the art. The cellular contents are contained within a vessel or a collection volume. In some aspects of the invention, cellular contents, such as genomic DNA, can be released from the cells by lysing the cells. Lysis can be achieved by, for example, heating the cells, or by the use of detergents or other chemical methods, or by a combination of these. However, any suitable lysis method known in the art can be used. For example, heating the cells at 72° C. for 2 minutes in the presence of Tween-20 is sufficient to lyse the cells. Alternatively, cells can be heated to 65° C. for 10 minutes in water (Esumi et al., Neurosci Res 60(4):439-51 (2008)); or 70° C. for 90 seconds in PCR buffer II (Applied Biosystems) supplemented with 0.5% NP-40 (Kurimoto et al., Nucleic Acids Res 34(5):e42 (2006)); or lysis can be achieved with a protease such as Proteinase K or by the use of chaotropic salts such as guanidine isothiocyanate (U.S. Publication No. 2007/0281313). Amplification of genomic DNA according to methods described herein can be performed directly on cell lysates, such that a reaction mix can be added to the cell lysates. Alternatively, the cell lysate can be separated into two or more volumes such as into two or more containers, tubes or regions using methods known to those of skill in the art with a portion of the cell lysate contained in each volume container, tube or region. Genomic DNA contained in each container, tube or region may then be amplified by methods described herein or methods known to those of skill in the art.

A nucleic acid used in the invention can also include native or non-native bases. In this regard a native deoxyribonucleic acid can have one or more bases selected from the group consisting of adenine, thymine, cytosine or guanine and a ribonucleic acid can have one or more bases selected from the group consisting of uracil, adenine, cytosine or guanine. Exemplary non-native bases that can be included in a nucleic acid, whether having a native backbone or analog structure, include, without limitation, inosine, xathanine, hypoxathanine, isocytosine, isoguanine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. A particular embodiment can utilize isocytosine and isoguanine in a nucleic acid in order to reduce non-specific hybridization, as generally described in U.S. Pat. No. 5,681,702.

As used herein, the term “primer” generally includes an oligonucleotide, either natural or synthetic, that is capable, upon forming a duplex with a polynucleotide template, of acting as a point of initiation of nucleic acid synthesis, such as a sequencing primer, and being extended from its 3′ end along the template so that an extended duplex is formed. The sequence of nucleotides added during the extension process is determined by the sequence of the template polynucleotide. Usually primers are extended by a DNA polymerase. Primers usually have a length in the range of between 3 to 36 nucleotides, also 5 to 24 nucleotides, also from 14 to 36 nucleotides. Primers within the scope of the invention include orthogonal primers, amplification primers, constructions primers and the like. Pairs of primers can flank a sequence of interest or a set of sequences of interest. Primers and probes can be degenerate or quasi-degenerate in sequence. Primers within the scope of the present invention bind adjacent to a target sequence. A “primer” may be considered a short polynucleotide, generally with a free 3′-OH group that binds to a target or template potentially present in a sample of interest by hybridizing with the target, and thereafter promoting polymerization of a polynucleotide complementary to the target. Primers of the instant invention are comprised of nucleotides ranging from 17 to 30 nucleotides. In one aspect, the primer is at least 17 nucleotides, or alternatively, at least 18 nucleotides, or alternatively, at least 19 nucleotides, or alternatively, at least 20 nucleotides, or alternatively, at least 21 nucleotides, or alternatively, at least 22 nucleotides, or alternatively, at least 23 nucleotides, or alternatively, at least 24 nucleotides, or alternatively, at least 25 nucleotides, or alternatively, at least 26 nucleotides, or alternatively, at least 27 nucleotides, or alternatively, at least 28 nucleotides, or alternatively, at least 29 nucleotides, or alternatively, at least 30 nucleotides, or alternatively at least 50 nucleotides, or alternatively at least 75 nucleotides or alternatively at least 100 nucleotides.

The expression “amplification” or “amplifying” refers to a process by which extra or multiple copies of a particular polynucleotide are formed.

The DNA amplified according to the methods described herein may be sequenced and analyzed using methods known to those of skill in the art. Determination of the sequence of a nucleic acid sequence of interest can be performed using a variety of sequencing methods known in the art including, but not limited to, sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (U.S. Ser. No. 12/120,541, filed May 14, 2008), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., using platforms such as Roche 454, Illumina Solexa, AB-SOLiD, Helicos, Polonator platforms and the like, can also be utilized. A variety of light-based sequencing technologies are known in the art (Landegren et al. (1998) Genome Res. 8:769-76; Kwok (2000) Pharmacogenomics 1:95-100; and Shi (2001) Clin. Chem. 47:164-172).

The amplified DNA can be sequenced by any suitable method. In particular, the amplified DNA can be sequenced using a high-throughput screening method, such as Applied Biosystems' SOLiD sequencing technology, or Illumina's Genome Analyzer. In one aspect of the invention, the amplified DNA can be shotgun sequenced. The number of reads can be at least 10,000, at least 1 million, at least 10 million, at least 100 million, or at least 1000 million. In another aspect, the number of reads can be from 10,000 to 100,000, or alternatively from 100,000 to 1 million, or alternatively from 1 million to 10 million, or alternatively from 10 million to 100 million, or alternatively from 100 million to 1000 million. A “read” is a length of continuous nucleic acid sequence obtained by a sequencing reaction.

“Shotgun sequencing” refers to a method used to sequence very large amount of DNA (such as the entire genome). In this method, the DNA to be sequenced is first shredded into smaller fragments which can be sequenced individually. The sequences of these fragments are then reassembled into their original order based on their overlapping sequences, thus yielding a complete sequence. “Shredding” of the DNA can be done using a number of difference techniques including restriction enzyme digestion or mechanical shearing. Overlapping sequences are typically aligned by a computer suitably programmed Methods and programs for shotgun sequencing a cDNA library are well known in the art.

The amplification and sequencing methods are useful in the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining the genomic DNA in order to determine whether an individual is at risk of developing a disorder and/or disease. Such assays can be used for prognostic or predictive purposes to thereby prophylactically treat an individual prior to the onset of the disorder and/or disease. Accordingly, in certain exemplary embodiments, methods of diagnosing and/or prognosing one or more diseases and/or disorders using one or more of expression profiling methods described herein are provided.

As used herein, the term “biological sample” is intended to include, but is not limited to, tissues, cells, biological fluids and isolates thereof, isolated from a subject, as well as tissues, cells and fluids present within a subject.

In certain exemplary embodiments, electronic apparatus readable media comprising one or more genomic DNA sequences described herein is provided. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon one or more expression profiles described herein.

As used herein, the term “electronic apparatus” is intended to include any suitable computing or processing apparatus or other device configured or adapted for storing data or information. Examples of electronic apparatuses suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising one or more expression profiles described herein.

A variety of software programs and formats can be used to store the genomic DNA information of the present invention on the electronic apparatus readable medium. For example, the nucleic acid sequence can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of data processor structuring formats (e.g., text file or database) may be employed in order to obtain or create a medium having recorded thereon one or more expression profiles described herein.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures and accompanying claims.

### EXAMPLE I

### General Protocol

The following general protocol is useful for whole genome amplification. A single cell is lysed in lysis buffer. The transposome library including transposome with a specific barcode pair, such as a unique barcode pair, and transposition buffer are added to the cell lysis which is mixed well and is incubated at 55° C. for 10 minutes. 1 mg/ml protease is added after the transposition to remove the transposase from binding to the single cell genomic DNA. Deepvent exo-DNA polymerase, dNTP, PCR reaction buffer and primers are added to the reaction mixture which is heated to 72° C. for 10 min to fill in the gap generated from the transposon insertion. The reaction mixture is loaded to the microfluidic device to form micro droplets. The droplets containing single cell genomic DNA template, DNA polymerase, dNTP, reaction buffer and primer are collected into PCR tubes. 40 to 60 cycles of PCR reaction are performed to amplify the single cell genomic DNA. The number of cycles is selected to drive the amplification reaction in the droplets to saturation. The droplets are lysed and the amplification products are purified for further analysis like high through put deep sequencing.

### EXAMPLE II

### Making a Transposome with Transposon DNA Homo Dimers

To make a transposomes with transposon DNA homodimers, (i.e. a transposome with the same barcode sequence on each transposon DNA), and accordingly, a library of transposomes with uniquely associated barcodes, a plurality of the transposon DNA containing a cleavage site (for example, a DNA nuclease cutting site), a priming site, a unique barcode sequence and a transposase binding site are linked to a single microparticle or bead, such that a single microparticle includes a plurality of transposon DNA with the same unique barcode sequence and no other barcode sequence.

As illustrated in FIG. 6, a plurality of barcoded transposon DNA as shown in FIG. 1 is attached to a microparticle, such as a bead, via a linker. A cleavage moiety or site is also provided so that the transposon DNA may be cleaved or otherwise removed from the microparticle.

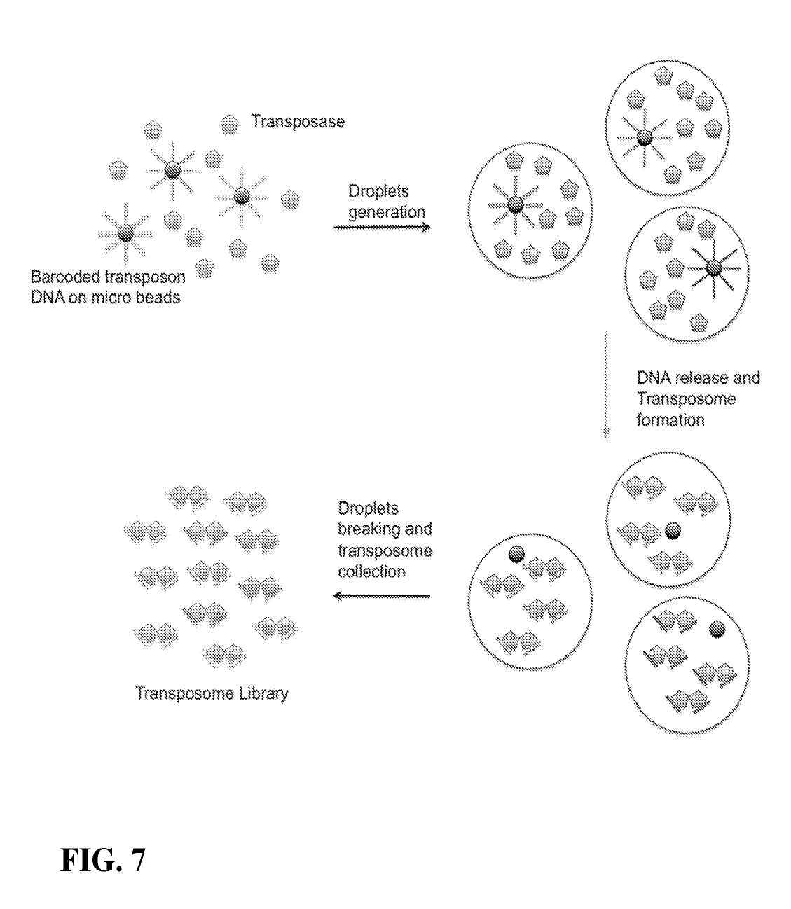

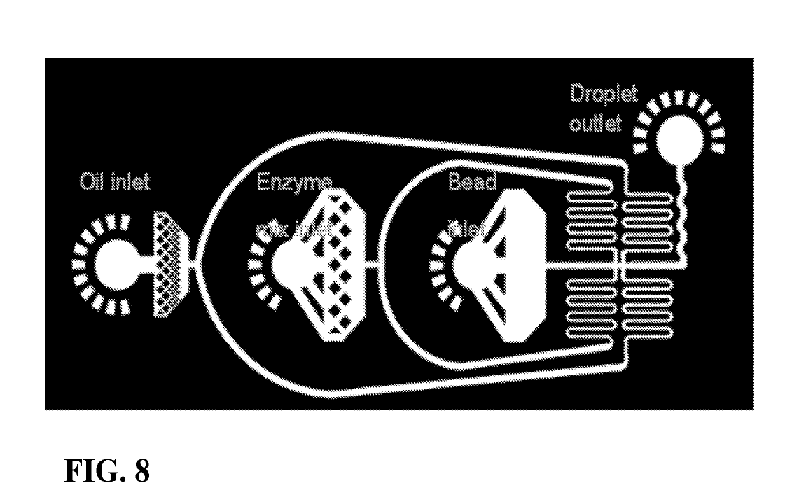

As illustrated in exemplary FIG. 7, a library of microparticles is created with each microparticle in the library having linked thereto a plurality of transposon DNA with its own unique barcode sequence. Millions of microparticles are contemplated with each microparticle having its own unique associated barcode sequence. The methods described herein provide for the making of millions of symmetrically indexed transposomes simultaneously and not separately, i.e. each transposome has its own unique associated barcode sequence because each transposon DNA of the transposome is identical and the number of transposomes produced in a single reaction volume is on the order of millions. Methods of making barcoded transposomes are described in WO2012/2061832, however such materials and methods are different from those described herein and result in a limited in the number of transposomes that can be made. According to one aspect, all of the transposon DNA on the same single micro particle of the library have the same barcode sequence, while each microparticle or substantially each microparticle in the library has its own unique associated barcode sequence, i.e. each microparticle includes transposon DNA with a barcode sequence that is different from each remaining microparticle in the library. According to one aspect, the number of transposon DNA molecules on a particular microparticle exceeds the number of transposase molecules which are to come into contact with the transposon DNA molecules to form transposomes. In this manner, each transposome will have two identical transposon DNA molecules, and so will also have the same barcode sequence in each of the two transposon DNA molecules. Having more transposon DNA molecules than there are transposase molecules ensures that no transposome lacks a transposon DNA molecule during formation of the transposomes within a microdroplet, for example. Accordingly, the presence of a transposome complex with two different transposon DNA molecules (and accordingly two different barcode sequences) is reduced or eliminated.

The beads are then loaded into micro droplets together with transposase and nuclease such that each microdroplet includes only one bead and, therefore, only one unique barcode. Within the microdroplet, the transposon DNAs are cleaved from the bead and transposomes having the same unique barcode sequence (i.e., transposon DNA homo dimers) are formed. The transposomes with homo dimeric transposon DNA are then collected after lysing or breaking the droplets to form the library of transposomes.

In particular, to make more than 1,000 transposomes each carrying its own uniquely associated barcode sequence, microparticles or beads and droplet microfluidics are utilized. M number of microparticles or beads that each carries DNA strands with a unique barcode are synthesized according to the methods described in Macosko et al. Cell 161 (5), 2015 hereby incorporated by reference in its entirety, such that there are on average n number of transposon DNA strands on each microparticle or bead that share the same barcode specifically associated with the microparticle or bead, and that each microparticle or bead has its own unique barcode sequence that differs from other microparticles or beads. Every transposon DNA strand is linked to the microparticle or bead via a linker molecule, and its sequence contains a cleavage site (for example, a single uracil nucleotide that can be cut by the USER™ Enzyme from New England Biolabs), a priming site, a unique barcode sequence and a transposase binding site, and all DNA strands on all beads or microparticles share the same sequence for cleavage site, the same sequence for priming site and the same sequence for transposase binding site. All microparticles or beads are then mixed with single-stranded DNA molecules of the same sequence that is complementary to the transposase binding site on DNA strands on beads or microparticles, so that partially double-stranded and partially single-stranded DNA molecules can be created on beads or microparticles as depicted in FIG. 6. Because the transposome inserts more efficiently to double stranded DNA than single stranded DNA, this partially single stranded DNA structure can prevent insertions between transposome molecules.

To make uniquely barcoded transposomes, each microparticle or bead is co-encapsulated into a droplet with a mixture of transposase enzyme and cutting enzyme (such as the USER™ Enzyme from New England Biolabs) using a flow-focusing microfluidic device such as the devices described in Macosko et al. Cell, 2015, 161 (5): p. 1202-14 and Klein et al. Cell, 2015, 161(5): p. 1187-1201 each of which is hereby incorporated by reference in its entirety, such that each droplet contains zero to one bead or microparticle. An exemplary flow circuit is illustrated in FIG. 8 which includes in fluid communication via microchannels an aqueous phase enzyme mix inlet, an aqueous phase bead inlet, a hydrophobic liquid inlet (referred to as an oil inlet), a combination zone for combining the enzyme mix with the beads, and a combination zone for combining the aqueous phase with the oil phase which is in further fluid communication by a microchannel to an emulsion droplet outlet region. The enzyme mix is combined with the beads and the combination is then formed into microdroplets with one bead per microdroplet.

A suitable hydrophobic phase is one that generates aqueous droplets when an aqueous media is introduced into the hydrophobic phase. Suitable oil phases are known to those of skill in the art in which an aqueous phase spontaneously results in aqueous droplets or isolated volumes or compartments surrounded by the oil phase. An exemplary hydrophobic phase includes a hydrophobic liquid, such as an oil, such as a fluorinated oil, such as 3-ethoxyperfluoro(2-methylhexane), and a surfactant. Surfactants are well known to those of skill in the art. An exemplary hydrophobic phase including a suitable oil and a surfactant is commercially available as QX200™ Droplet Generation Oil for Evagreen (Bio-Rad), a hydrophobic surfactant-containing liquid that does not mix with aqueous solution or adversely affect biochemical reactions in aqueous solution, 008-FluoroSurfactant in HFE 7500 (RAN Biotechnologies), Pico-Surf™ 1 (Dolomite Microfluidics), Proprietary Oil Surfactants (RainDance Technologies), fluorosurfactants described in fluorinated oils discussed in Mazutis, L., et al. Single-cell analysis and sorting using droplet-based microfluidics, Nature Protocols, 2013, 8, p. 870-891, and other surfactants described in Baret, J.-C., Lab on a Chip, 2012, 12, p. 422-433 each of which is hereby incorporated by reference in its entirety.

When the oil phase and the aqueous phase are combined in the combination region or the emulsion droplet outlet region, the aqueous phase will spontaneously form droplets surrounded by the oil phase. According to one aspect, a flush volume of a hydrophobic fluid, such as an oil which may not contain a surfactant as none is needed for a flush volume, upstream of the aqueous phase either within the microfluidic design or within a syringe or injector used to input the aqueous bead phase or aqueous enzyme mix phase into the microfluidic design is used to displace any aqueous phase that may otherwise occupy a dead volume to minimize loss of original aqueous phase introduced into the microfluidic chip design. Useful microfluidic chip designs can be created using AutoCAD software (Autodesk Inc.) and can be printed by CAD Art Services Inc. into a photomask for microfluidic fabrication. Molds or masters can be created using conventional techniques as described in Mazutis et al. Nature Protocols 8 (5), 2013 hereby incorporated by reference in its entirety. Microfluidic chips can be made from the master by curing uncured polydimethyl siloxane (PDMS) (Dow Corning Sylgard 184) poured onto the master and heated to curing to create a surface with trenches or circuits. Inlet and outlet holes are created and the cured surface with the circuits is placed against a glass slide and secured to create the microchannels and the microfluidic chip. Before use, the interior of the microfluidic chip can be treated with a compound for improving the hydrophobicity of the interior of the microfluidic chip and washed to remove potential contamination.

According to one aspect, general methods known to those of skill in the art are used to create droplets where each droplet includes a single bead or no bead. The enzyme mix in aqueous media and the beads in aqueous media are combined and the combination is introduced into oil which results in droplets where the number of droplets exceeds the number of beads such that a single bead is isolated within a single droplet along with sufficient enzymes.

Within each droplet, the n number of transposon DNA molecules attached to the microparticle are cut from the microparticle or bead by the cutting enzyme, and spontaneously assemble with transposase monomers within the microparticle into around n/2 number of transposomes, each of which is composed of two transposase monomers and two transposon DNA molecules with the same barcode, as depicted in FIG. 7. The number of barcodes, which is the number of encapsulated microparticles or beads (i.e. M), and the average number of transposomes in a droplet, which is half of the average number of DNA strands on each microparticle or bead (i.e. half of n), are scaled such that transposomes with statistically unique barcodes can be obtained for cutting and insertion or addition of transposon DNA at the cut site, i.e. to each end of an adjacent genomic DNA fragment.

Transposomes with effectively unique barcodes are obtained by pooling all the M times n/2 number of transposomes by lysing the droplets, i.e. demulsification, and collecting the transposomes, and having a small portion, significantly less than 1/(n/2) of the total amount, of the pool of transposomes (having M barcodes and on average n/2 copies of each barcode) insert into the genome, so that the chance of having two or more transposomes with the same barcode insert into the genome is statistically minute. Lysing of the droplets or demulsification can be accomplished by adding perfluorooctanol (TCI Chemicals) to the droplets and after shaking by hand or vortexing and centrifugation, all droplets are lysed and aqueous solution containing the transposomes is collected.

As a non-limiting example, to assemble a human genome with around 6,000,000,000 base pairs, 1,000,000 transposomes with unique barcodes are needed for insertion into the genome, assuming an average insertion length of 6000 bp, so M is at least 10⁶, which can be 10⁷, for example. Given that a typical microparticle or bead can bear around 10⁸ DNA molecules as explained in Macosko et al. Cell 161 (5), 2015, n=10⁸ is a reasonable estimate. As a result, if M=10⁷ microparticles or beads are used to make 5×10¹⁴ (M times n/2) number of barcoded transposomes, 1/166667 of the total pool of transposomes can be taken and added to the genomic DNA, and around 1/3000 of the added transposomes can insert into the genome, so the final number of transposomes that insert into the genome is estimated to be 5×10¹⁴×1/166667×1/3000, which is approximately 1,000,000. In this example, the transposomes that insert into the genome is approximately 1/500,000,000 (1/166667 times 1/3000), which is significantly less than 1/(n/2), so the chance of having two identical barcodes that insert into the genome is statistically minute. In short, to assemble a human genome using an average insertion length of 6000 bp, 10 million uniquely barcoded beads can be used for making barcoded transposomes, and ion this example 1/166667 of the total transposomes need to be added to the genomic DNA for insertion.

In one embodiment, the cutting site for each DNA strand on a microparticle or bead can be a site that can be cleaved upon UV light exposure, such as the cleavage site described in Klein, A. M., et al. Droplet barcoding for single-cell transcriptomics applied to embryonic stem cells. Cell, 2015. 161(5): p. 1187-1201, which is hereby incorporated by reference in its entirety. The aqueous phase for enzyme mix in this example may not contain cutting enzymes for cleaving DNA strands from the microparticles or beads.

In another embodiment, the barcoded beads (or particles or microparticles) may be porous beads in such a way that DNA molecules can bind on the material or within the porous network of the material. The buffer for the enzyme mix can be chosen so that once a bead is co-encapsulated into a droplet with the enzyme mix, the DNA bound on the bead or within the pores of the bead can be released from the bead and subsequently assemble with transposase monomers into transposomes within the droplet. Examples of materials and methods that can carry and release DNA in a controlled manner depending on buffer conditions include the GemCode™ particles (10× Genomics), include the spin columns in nucleic acid purification kits such as the DNA Clean & Concentrator™-5 (Zymo Research), Monarch Nucleic Acid Purification Kits (New England Biolabs), and QIAquick PCR Purification Kit (Qiagen), and include the materials and methods described in Boom, R. et al. Rapid and simple method for purification of nucleic acids. Journal of Clinical Microbiology, 1990, 28(3), p. 495-503; Chen, C. W. and Thomas Jr., C. A. Recovery of DNA segments from agarose gels. Analytical Biochemistry, 1980, 101(2), p. 339-341; and Tian, H., et al. Evaluation of silica resins for direct and efficient extraction of DNA from complex biological matrices in a miniaturized format. Analytical Biochemistry, 2000, 283, p. 175-191 each of which is hereby incorporated by reference in its entirety.

In some aspect, the barcoded particles may be replaced by barcoded droplets which have been exemplified and described in Lan, F., et al. Droplet barcoding for massively parallel single-molecule deep sequencing. Nature Communications, 2016, 7:11784 which is hereby incorporated by reference in its entirety. The enzyme mix can then be introduced into the barcoded droplets using pico-injection or droplet merging methods described in Abate, A., et al. High-throughput injection with microfluidics using picoinjectors. Proceedings of the National Academy of Sciences of the united States of America, 2010, 107(45), p. 19163-19166; Lan, F., et al. Droplet barcoding for massively parallel single-molecule deep sequencing. Nature Communications, 2016, 7:11784; and Rhee, M., et al. Pressure stabilizer for reproducible picoinjection in droplet microfluidic systems. Lab on a Chip, 2014, 14(23), p. 4533-4539 each of which is hereby incorporated by reference in its entirety. Within each droplet, the introduced transposase monomers can then be assemble with the transposon DNA molecules with droplet-specific barcode into transposomes. All the droplets can then be lysed so that barcoded transposomes can be pooled for insertion into genomic DNA with barcoded annotation.

According to one aspect, the transposomes with the transposon DNA sequences described herein may be synthesized in separate compartments that are not created using droplet microfluidics; examples of such platforms, instruments, materials or methods include multi-well plates, high-throughput synthesizers, microarrays, microwells, microreactors or other compartmentalization methods such as those described in Sims, P. A., et al., Fluorogenic DNA sequencing in PDMS microreactors. Nature Methods, 2011, 8(7), p. 575-580; Gole, J., et al., Massively parallel polymerase cloning and genome sequencing of single cells using nanoliter microwells. Nature Biotechnology, 2013, 31(12), p. 1126-1132; Leung K., et al., Robust high-performance nanoliter-volume single-cell multiple displacement amplification on planar substrates. Proceedings of the National Academy of Sciences of the United States of America. 2016, 113(30), p. 8484-8489; and Zarzar, L. D., et al., Dynamically reconfigurable complex emulsions via tunable interface tensions. Nature, 2015, 518, p. 520-524 each of which is hereby incorporated by reference in its entirety.

### EXAMPLE III

### Cell Lysis

A cell is selected, cut from a culture dish, and dispensed in a tube using a laser dissection microscope (LMD-6500, Leica) as follows. The cells are plated onto a membrane-coated culture dish and observed using bright field microscopy with a 10× objective (Leica). A UV laser is then used to cut the membrane around an individually selected cell such that it falls into the cap of a PCR tube. The tube is briefly centrifuged to bring the cell down to the bottom of the tube. 3-5 μl lysis buffer (30 mM Tris-Cl PH 7.8, 2 mM EDTA, 20 mM KCl, 0.2% Triton X-100, 500 μg/ml Qiagen Protease) is added to the side of the PCR tube and span down. The captured cell is then thermally lysed using the using following temperature schedule on PCR machine: 50° C. 3 hours, 75° C. 30 minutes. Alternatively, mouth pipette a single cell into a low salt lysis buffer containing EDTA and protease such as QIAGEN protease (QIAGEN) at a concentration of 10-5000 μg/mL. The incubation condition varies based on the protease that is used. In the case of QIAGEN protease, the incubation would be 37-55° C. for 1-4 hrs. The protease is then heat inactivated up to 80° C. and further inactivated by specific protease inhibitors such as 4-(2-Aminoethyl) benzenesulfonyl fluoride hydrochloride (AEBSF) or phenylmethanesulfonyl fluoride (PMSF) (Sigma Aldrich). The cell lysis is preserved at −80° C.

### EXAMPLE IV

### Transposition

The single cell lysis and the transposome library are mixed in a buffer system containing 1-100 mM Mg²⁺ and optionally 1-100 mM Mn²⁺ or Co²⁺ or Ca²⁺ as well and incubate at 37-55° C. for 5-240 minutes. The reaction volume varies depending on the cell lysis volume. The amount of transposome library added in the reaction could be readily tuned depending on the desired fragmentation size. The transposition reaction is stopped by chelating Mg²⁺ using EDTA and optionally EGTA or other chelating agents for ions. Optionally, short double stranded DNA could be added to the mixture as a spike-in. The residue transposome is inactivated by protease digestion such as QIAGEN protease at a final concentration 1-500 μg/mL at 37-55° C. for 10-60 minutes. The protease is then inactivated by heat and/or protease inhibitor, such as AEBSF.

### EXAMPLE V

### Gap Filling

After transposition and transposase removal, a PCR reaction mixture including Mg²⁺, dNTP mix, primers and a thermal stable DNA polymerase such as Deepvent exo-DNA polymerase (New England Biolabs) is added to the solution at a suitable temperature and for a suitable time period to fill the 9 bp gap left by the transposition reaction. The gap filling incubation temperature and time depends on the specific DNA polymerase used. After the reaction, the DNA polymerase is optionally inactivated by heating and/or protease treatment such as QIAGEN protease. The protease, if used, is then inactivated by heat and/or protease inhibitor.

### EXAMPLE VI

### DNA Fragment Amplification

According to one aspect, general methods known to those of skill in the art are used to amplify a DNA fragment. The gap filled double stranded products from the above example including the DNA fragments with primer binding sites are added to PCR reaction reagents in an aqueous medium. The aqueous medium is then subject to PCR conditions to PCR amplify each DNA fragment.

### EXAMPLE VII

### Sequencing of DNA Fragment Amplicons and De Novo Genome Assembly Using Barcodes

According to one aspect, the fragments are sequenced using methods known to those of skill in the art and the sequences are stored in computer readable memory. The sequences then can be compared and fragments having matching barcode sequences can be identified. Fragments having matching barcode sequences are then identified as having been sequences that were adjacent to each other in the original genomic DNA sequence. Two or more adjacent sequences can then be computationally linked together, i.e. in silico using computer software, to create longer sequence fragments of the original genomic DNA. In this aspect, the disclosure provides methods of de novo assembly of fragments of genomic DNA created using transposome barcodes to create longer fragments.

According to one aspect, each end of every genomic DNA fragment has a gap-filled sequence in addition to the transposase binding site sequence, barcode sequence and the priming sequence. The gap filled sequence can serve as a second set of barcodes for chaining different fragments into longer genomic sequences because it is a duplicated sequence shared by two fragments cut by a transposome. For example, it is known that when a Tn5 transposome inserts into the double-stranded genomic DNA template, it leaves a single stranded 9 bp gap at each of the two ends of the insertion site, as shown in FIG. 3, and both 9 bp gaps across the same insertion site will share the same sequence after the gap filling step (also known as a gap extension step) that is shown in FIG. 4. Such a 9 bp sequence that is duplicated across the insertion site can serve as an additional barcode for chaining fragments for de novo assembly, which is very helpful when insertions of two transposomes carrying the same barcode sequence happen.

According to one aspect, fragments are de novo assembled in silico by matching barcode sequences to recreate the original genomic DNA sequence, such as whole genomic DNA. After chaining all the fragments using the barcode information, the chained, linked or assembled continuous or contiguous genomic sequence made up of fragments, also known as a “contig”, may be compared with or matched to another contig that share similar or identical sequence from a homologous chromosome, and by matching contigs from homologous chromosomes, the genomic sequences or contigs can be further linked into longer sequences or contigs that are ultimately assembled into the entire genome. The de novo assembly methods known to those of skill in the art include the overlap-layout-consensus (OLC), de Bruijn, the string graph approaches and other assembly algorithms reviewed in Chaisson, M. J. P. et al., Genetic variation and the de novo assembly of human genomes. Nature Review Genetics, 2015. 16: p. 627-640 which is hereby incorporated by reference in its entirety for all purposes.

According to one aspect, genomes from two, three, four or more daughter cells or identical cells can be individually fragmented and amplified with barcoded annotation, sequenced, separately assembled using the aforementioned methods to effectively provide substantial homologous chromosome pairs for cross-referencing in order to arrive at a unique de novo assembled genome map. These methods may be combined with the de novo assembly approaches that utilize overlapping regions between homologs such as SSAKE, SHARCGS, VCAKE, Newbler, Celera Assembler, Euler, Velvet, ABySS, AllPaths, and SOAPdenovo reviewed in Miller et al. Genomics, 95(6), 2010; and the algorithms described in Chaisson et al. Nature Reviews Genetics, 16, 2015, each of which is hereby incorporated by reference in its entirety, to provide substantial homologous overlaps for high-quality whole genome de novo assembly.

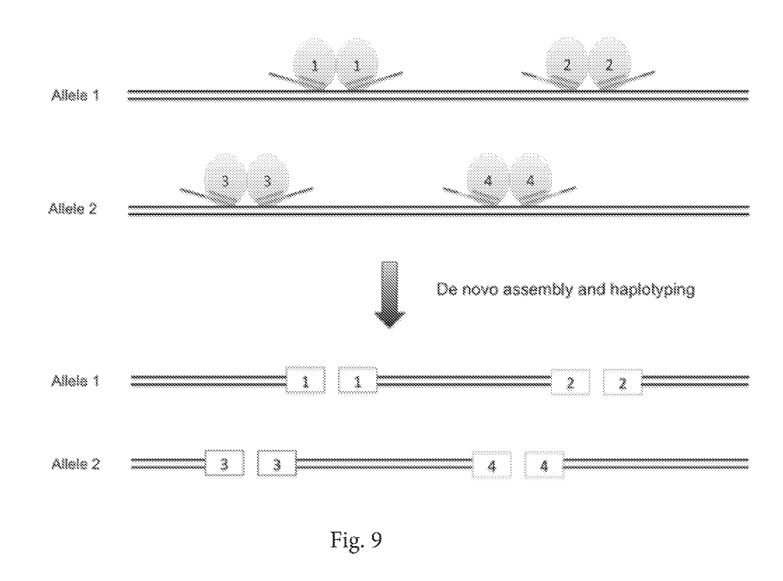

When the target genomic DNA is from a single cell with more than one ploidy, the de novo assembly of the genome can also achieve haplotyping as illustrated in FIG. 9. Ploidy is the number of sets of chromosomes in a cell. For example, human somatic cells have two sets of homologous copies of each chromosome. The two copies, or alleles, are from the father and the mother separately and are two physically separate DNA molecules in the cell. Because the two copies are not joined together and use of transposomes for transposition, i.e. insertion of the transposon DNA and production of fragments, happens independently for each separate copy, any part of one copy does not share the same insertion site with any part of the other copy, so fragments from one copy do not contain barcodes that can be matched to any barcode on fragments of the other copy, and so fragments from one copy will not be linked or chained to those from the other copy. For example, as illustrated in FIG. 9, transposomes 1 and 2 insert transposon DNA into the first allele and transposomes 3 and 4 insert transposon DNA into the second allele. After the independent transposome initiated insertion of transposon DNA for each separate allele and after amplification, sequencing, and de novo assembly using the methods described herein, the two alleles are assembled separately and the final assembled product is a haplotype-resolved genome. This is because the fragments of Allele 1 do not share the same barcode with any fragment from Allele 2. So fragments from Allele 1 will not be linked or chained to those from Allele 2, and fragments within each allele can be linked or chained independent of any information from Allele 2, and vice versa. Accordingly, the resulting de novo assembly will result in longer chains in chained sequence and a whole chromosome assembly from the same allele and therefore, the genomic DNA is haplotype resolved. In contrast, when a human genome is assembled by shot-gun sequencing, it is taken as a haploid genome because the two alleles are almost identical and cannot be distinguished. Using the transposome method described herein, the two sets of chromosomes are assembled separately as illustrated in FIG. 9 because of the unique barcode sequences associated with each allele. The method allows distinguishing allele 1 from allele 2 by linking all the allele 1 fragments one by one by matching barcodes, and by linking all the allele 2 fragments one by one by matching barcodes. The assembling of the unique barcodes results in the de novo assembly of the separate alleles resulting in haplotype resolution.

### EXAMPLE XI

### Kits

The materials and reagents required for the disclosed amplification method may be assembled together in a kit. The kits of the present disclosure generally will include at least the transposome (consists of transposase enzyme and transposon DNA), nucleotides, and DNA polymerase necessary to carry out the claimed method along with primer sets as needed. In a preferred embodiment, the kit will also contain directions for amplifying DNA from DNA samples. Exemplary kits are those suitable for use in amplifying whole genomic DNA. In each case, the kits will preferably have distinct containers for each individual reagent, enzyme or reactant. Each agent will generally be suitably aliquoted in their respective containers. The container means of the kits will generally include at least one vial or test tube. Flasks, bottles, and other container means into which the reagents are placed and aliquoted are also possible. The individual containers of the kit will preferably be maintained in close confinement for commercial sale. Suitable larger containers may include injection or blow-molded plastic containers into which the desired vials are retained. Instructions are preferably provided with the kit.

### EXAMPLE XII

### Embodiments

The disclosure provides a method of making a transposome library including the steps of attaching a plurality of transposon DNA to each of a plurality of microparticles, wherein all transposon DNA attached to a single microparticle includes a common unique barcode sequence associated with the single microparticle, such that each microparticle of the plurality has a unique associated barcode sequence, combining the plurality of microparticles with the transposon DNA attached thereto with transposase and a cleavage enzyme to form an aqueous mixture, combining the aqueous mixture with an oil phase such that a plurality of microdroplets are formed wherein each microparticle of the plurality is isolated within a corresponding single microdroplet along with the transposase and the cleavage enzyme, for each corresponding single microdroplet, cleaving the plurality of transposon DNA from the microparticle within the corresponding single microdroplet and forming a plurality of transposomes within the microdroplet with each transposome within the microdroplet having two transposon DNA with the common unique barcode sequence, lysing each microdroplet of the plurality of microdroplets, and collecting the transposomes to create the transposome library. According to one aspect, the transposome library includes greater than 1,000 transposomes. According to one aspect, the transposome library includes greater than 10,000 transposomes. According to one aspect, the transposome library includes greater than 100,000 transposomes. According to one aspect, the transposome library includes greater than 1,000,000 transposomes. According to one aspect, the transposome library includes greater than 2,000,000 transposomes. According to one aspect, the transposome library includes greater than 3,000,000 transposomes. According to one aspect, the transposome library includes greater than 4,000,000 transposomes. According to one aspect, the transposome library includes greater than 5,000,000 transposomes. According to one aspect, the transposome library includes greater than 10,000,000 transposomes. According to one aspect, the method further includes taking a portion of the transposome library to form a reagent transposome library wherein each transposome of the reagent transposome library has a unique associated barcode sequence. According to one aspect, the method further includes taking a portion of the transposome library to form a reagent transposome library wherein substantially all transposomes within the reagent transposome library have a unique associated barcode sequence. According to one aspect, each transposon DNA includes a specific primer binding site and a double stranded transposase binding site. According to one aspect, the transposon DNA includes a double-stranded transposase binding site and an overhang, wherein the overhang includes a barcode sequence and a primer binding site at the 5′ end of the overhang. According to one aspect, each transposon DNA is attached to a corresponding microparticle by a linker and a cleavage site. According to one aspect, each transposon DNA includes a 5′ overhang and is attached at its corresponding 5′ end to a corresponding microparticle by a linker and a cleavage site. According to one aspect, the transposase is Tn5 transposase, Mu transposase, Tn7 transposase or IS5 transposase. According to one aspect, the oil phase includes a surfactant. According to one aspect, the plurality of microdroplets within the oil phase are created by combining the aqueous mixture with the oil phase in a manner to create more microdroplets than there are microparticles. According to one aspect, the plurality of microdroplets within the oil phase are created by combining the aqueous mixture with the oil phase in a manner to create more microdroplets than there are microparticles and wherein the plurality of microdroplets are spontaneously created. According to one aspect, the plurality of microdroplets within the oil phase are created by combining the oil phase and the aqueous media within a microfluidic chip.

According to one aspect, the plurality of microdroplets are lysed by a demulsification agent.

The disclosure provides a method of de novo genomic DNA assembly including the steps of contacting genomic DNA with a library of transposomes with each transposome of the library having its own unique associated barcode sequence, wherein each transposome of the library includes a transposase and a transposon DNA homo dimer, wherein each transposon DNA of the homo dimer includes a transposase binding site, a unique barcode sequence and a primer binding site, wherein the library of transposomes bind to target locations along the genomic DNA and the transposase cleaves the genomic DNA into a plurality of double stranded genomic DNA fragments representing a genomic DNA fragment library, with each double stranded genomic DNA fragment includes one member of a unique barcode sequence pair on each end of the genomic DNA fragment, gap filling a gap between the transposon DNA and the genomic DNA fragment to form a library of double stranded genomic DNA fragment extension products having primer binding sites at each end, amplifying the double stranded genomic DNA fragment extension products to produce amplicons, sequencing the amplicons, and computationally linking together the amplicons by matching barcodes so as to de novo assemble the genomic DNA. According to one aspect, the genomic DNA is whole genomic DNA obtained from a single cell. According to one aspect, the transposase is Tn5 transposase, Mu transposase, Tn7 transposase or IS5 transposase. According to one aspect, the transposon DNA includes a double-stranded 19 bp Tnp binding site and an overhang, wherein the overhang includes a barcode sequence and a primer binding site at the 5′ end of the overhang. According to one aspect, bound transposases are removed from the double stranded fragments before gap filling and extending of the double stranded genomic DNA fragments. According to one aspect, the transposases are Tn5 transposases each complexed with a transposon DNA, wherein the transposon DNA includes a double-stranded 19 bp Tnp binding site and an overhang, wherein the overhang includes a barcode sequence and a primer binding site. According to one aspect, the genomic DNA is from a prenatal cell. According to one aspect, the genomic DNA is from a cancer cell. According to one aspect, the genomic DNA is from a circulating tumor cell. According to one aspect, the genomic DNA is from a single prenatal cell. According to one aspect, the genomic DNA is from a single cancer cell. According to one aspect, the genomic DNA is from a single circulating tumor cell. According to one aspect, the primer binding site is a specific PCR primer binding site. According to one aspect, the de novo assembly is a haplotype-resolved de novo assembly. According to one aspect, the haplotype-resolved de novo assembly is on a human leukocyte antigen region, V(D)J recombination region or other regions of human single cells.

The disclosure provides a method of de novo genomic DNA assembly including the steps of creating a plurality of aqueous microdroplets within a nonaqueous phase, wherein each microdroplet includes a plurality of transposomes formed within the microdroplet, with all transposomes having two transposases and two identical transposon DNA, with each transposon DNA having a transposase binding site, a barcode sequence and a primer binding site, releasing the plurality of transposomes from each microdroplet and collecting the released transposomes into a transposome library, forming a reagent transposome library within a reaction volume wherein substantially all or all transposomes within the reagent transposome library have a unique associated barcode sequence, contacting genomic DNA with the reagent transposome library within the reaction volume wherein the transposomes bind to target locations along the genomic DNA and the transposase cleaves the genomic DNA into a plurality of double stranded genomic DNA fragments representing a genomic DNA fragment library, with each double stranded genomic DNA fragment including one member of a unique barcode sequence pair on each end of the genomic DNA fragment, gap filling a gap between the transposon DNA and the genomic DNA fragment to form a library of double stranded genomic DNA fragment extension products having primer binding sites at each end within the reaction volume, amplifying the double stranded genomic DNA fragment extension products to produce amplicons within the reaction volume, sequencing the amplicons within the reaction volume, and computationally linking together the amplicons by matching barcodes so as to de novo assemble the genomic DNA. According to one aspect, the reagent transposome library includes greater than 1,000 transposomes. According to one aspect, the reagent transposome library includes greater than 10,000 transposomes. According to one aspect, the reagent transposome library includes greater than 100,000 transposomes. According to one aspect, the reagent transposome library includes greater than 1,000,000 transposomes. According to one aspect, the reagent transposome library includes greater than 2,000,000 transposomes. According to one aspect, the reagent transposome library includes greater than 3,000,000 transposomes. According to one aspect, the reagent transposome library includes greater than 4,000,000 transposomes. According to one aspect, the reagent transposome library includes greater than 5,000,000 transposomes. According to one aspect, the reagent transposome library includes greater than 10,000,000 transposomes. According to one aspect, the genomic DNA is whole genomic DNA obtained from a single cell. According to one aspect, the transposase is Tn5 transposase, Mu transposase, Tn7 transposase or IS5 transposase. According to one aspect, the transposon DNA includes a double-stranded 19 bp Tnp binding site and an overhang, wherein the overhang includes a barcode sequence and a primer binding site at the 5′ end of the overhang. According to one aspect, bound transposases are removed from the double stranded fragments before gap filling and extending of the double stranded genomic DNA fragments. According to one aspect,the transposases are Tn5 transposases each complexed with a transposon DNA, wherein the transposon DNA includes a double-stranded 19 bp Tnp binding site and an overhang, wherein the overhang includes a barcode sequence and a primer binding site. According to one aspect, the genomic DNA is from a prenatal cell. According to one aspect, the genomic DNA is from a cancer cell. According to one aspect, the genomic DNA is from a circulating tumor cell. According to one aspect, the genomic DNA is from a single prenatal cell. According to one aspect, the genomic DNA is from a single cancer cell. According to one aspect, the genomic DNA is from a single circulating tumor cell. According to one aspect, the primer binding site is a specific PCR primer binding site.

The disclosure provides a method of de novo genomic DNA assembly including the steps of contacting transposases with a plurality of transposon DNA within physically separated reaction chambers to form transposomes within each physically separated reaction chamber, wherein each transposon DNA includes a common transposase binding site, a common primer binding site and a barcode sequence, wherein the barcode sequence is the same for all transposon DNA within the same reaction chamber, but different from transposon DNA within other reaction chambers, collecting the transposomes from each reaction chamber and mixing all the transposomes to form a transposome library, forming a reagent transposome library within a reaction volume wherein substantially all or all transposomes within the reagent transposome library have a unique associated barcode sequence, contacting genomic DNA with the reagent transposome library within the reaction volume wherein the transposomes bind to target locations along the genomic DNA and the transposase cleaves the genomic DNA into a plurality of double stranded genomic DNA fragments representing a genomic DNA fragment library, with each double stranded genomic DNA fragment including one member of a unique barcode sequence pair on each end of the genomic DNA fragment, gap filling a gap between the transposon DNA and the genomic DNA fragment to form a library of double stranded genomic DNA fragment extension products having primer binding sites at each end within the reaction volume, amplifying the double stranded genomic DNA fragment extension products to produce amplicons within the reaction volume, sequencing the amplicons within the reaction volume, and computationally linking together the amplicons by matching barcodes so as to de novo assemble the genomic DNA. According to one aspect, the reaction chambers are tubes, multi-well plates, micro-array chips, micro-wells, micro-reactors, micro-droplets, micro-particles hydrogel or other compartmentalization methods.

What is claimed is:

1. A method of making a transposome library comprising
attaching a plurality of transposon DNA to each of a plurality of microparticles, wherein all transposon DNA attached to a single microparticle includes a common unique barcode sequence associated with the single microparticle, such that each microparticle of the plurality has a unique associated barcode sequence,
combining the plurality of microparticles with the transposon DNA attached thereto with transposase and a cleavage enzyme to form an aqueous mixture,
combining the aqueous mixture with an oil phase such that a plurality of microdroplets are formed wherein each microparticle of the plurality is isolated within a corresponding single microdroplet along with the transposase and the cleavage enzyme,
for each corresponding single microdroplet, cleaving the plurality of transposon DNA from the microparticle within the corresponding single microdroplet and forming a plurality of transposomes within the microdroplet with each transposome within the microdroplet having two transposon DNA with the common unique barcode sequence,
lysing each microdroplet of the plurality of microdroplets, and
collecting the transposomes to create the transposome library.

2. The method of claim 1 wherein the transposome library includes greater than 1,000 transposomes.

3. The method of claim 1 wherein the transposome library includes greater than 10,000 transposomes.

4. The method of claim 1 wherein the transposome library includes greater than 100,000 transposomes.

5. The method of claim 1 wherein the transposome library includes greater than 1,000,000 transposomes.

6. The method of claim 1 wherein the transposome library includes greater than 2,000,000 transposomes.

7. The method of claim 1 wherein the transposome library includes greater than 3,000,000 transposomes.

8. The method of claim 1 wherein the transposome library includes greater than 4,000,000 transposomes.

9. The method of claim 1 wherein the transposome library includes greater than 5,000,000 transposomes.

10. The method of claim 1 wherein the transposome library includes greater than 10,000,000 transposomes.

11. The method of claim 1 further comprising taking a portion of the transposome library to form a reagent transposome library wherein each transposome of the reagent transposome library has a unique associated barcode sequence.

12. The method of claim 1 further comprising taking a portion of the transposome library to form a reagent transposome library wherein substantially all transposomes within the reagent transposome library have a unique associated barcode sequence.

13. The method of claim 1 wherein each transposon DNA includes a specific primer binding site and a double stranded transposase binding site.

14. The method of claim 1 wherein the transposon DNA includes a double-stranded transposase binding site and an overhang, wherein the overhang includes a barcode sequence and a primer binding site at the 5′ end of the overhang.

15. The method of claim 1 wherein each transposon DNA is attached to a corresponding microparticle by a linker and a cleavage site.

16. The method of claim 1 wherein each transposon DNA includes a 5′ overhang and is attached at its corresponding 5′ end to a corresponding microparticle by a linker and a cleavage site.

17. The method of claim 1 wherein the transposase is Tn5 transposase, Mu transposase, Tn7 transposase or IS5 transposase.

18. The method of claim 1 wherein the oil phase includes a surfactant.

19. The method of claim 1 wherein the plurality of microdroplets within the oil phase are created by combining the aqueous mixture with the oil phase in a manner to create more microdroplets than there are microparticles.

20. The method of claim 1 wherein the plurality of microdroplets within the oil phase are created by combining the aqueous mixture with the oil phase in a manner to create more microdroplets than there are microparticles and wherein the plurality of microdroplets are spontaneously created.

21. The method of claim 1 wherein the plurality of microdroplets within the oil phase are created by combining the oil phase and the aqueous media within a microfluidic chip.

22. The method of claim 1 wherein the plurality of microdroplets are lysed by a demulsification agent.

23. A method of de novo genomic DNA assembly comprising
contacting genomic DNA with a library of transposomes with each transposome of the library having its own unique associated barcode sequence, wherein each transposome of the library includes a transposase and a transposon DNA homo dimer, wherein each transposon DNA of the homo dimer includes a transposase binding site, a unique barcode sequence and a primer binding site, wherein the library of transposomes bind to target locations along the genomic DNA and the transposase cleaves the genomic DNA into a plurality of double stranded genomic DNA fragments representing a genomic DNA fragment library, with each double stranded genomic DNA fragment includes one member of a unique barcode sequence pair on each end of the genomic DNA fragment,
gap filling a gap between the transposon DNA and the genomic DNA fragment to form a library of double stranded genomic DNA fragment extension products having primer binding sites at each end,
amplifying the double stranded genomic DNA fragment extension products to produce amplicons,
sequencing the amplicons, and
computationally linking together the amplicons by matching barcodes so as to de novo assemble the genomic DNA.

24. The method of claim 23 wherein the genomic DNA is whole genomic DNA obtained from a single cell.

25. The method of claim 23 wherein the transposase is Tn5 transposase, Mu transposase, Tn7 transposase or IS5 transposase.

26. The method of claim 23 wherein the transposon DNA includes a double-stranded 19 bp Tnp binding site and an overhang, wherein the overhang includes a barcode sequence and a primer binding site at the 5′ end of the overhang.

27. The method of claim 23 wherein bound transposases are removed from the double stranded fragments before gap filling and extending of the double stranded genomic DNA fragments.

28. The method of claim 23 wherein the transposases are Tn5 transposases each complexed with a transposon DNA, wherein the transposon DNA includes a double-stranded 19 bp Tnp binding site and an overhang, wherein the overhang includes a barcode sequence and a primer binding site.

29. The method of claim 23 wherein the genomic DNA is from a prenatal cell.

30. The method of claim 23 wherein the genomic DNA is from a cancer cell.

31. The method of claim 23 wherein the genomic DNA is from a circulating tumor cell.

32. The method of claim 23 wherein the genomic DNA is from a single prenatal cell.

33. The method of claim 23 wherein the genomic DNA is from a single cancer cell.

34. The method of claim 23 wherein the genomic DNA is from a single circulating tumor cell.

35. The method of claim 23 wherein the primer binding site is a specific PCR primer binding site.

36. The method of claim 23 wherein the de novo assembly is a haplotype-resolved de novo assembly.

37. A method of de novo genomic DNA assembly comprising
creating a plurality of aqueous microdroplets within a nonaqueous phase, wherein each microdroplet includes a plurality of transposomes formed within the microdroplet, with all transposomes having two transposases and two identical transposon DNA, with each transposon DNA having a transposase binding site, a barcode sequence and a primer binding site,
releasing the plurality of transposomes from each microdroplet and collecting the released transposomes into a transposome library,
forming a reagent transposome library within a reaction volume wherein substantially all or all transposomes within the reagent transposome library have a unique associated barcode sequence,
contacting genomic DNA with the reagent transposome library within the reaction volume wherein the transposomes bind to target locations along the genomic DNA and the transposase cleaves the genomic DNA into a plurality of double stranded genomic DNA fragments representing a genomic DNA fragment library, with each double stranded genomic DNA fragment including one member of a unique barcode sequence pair on each end of the genomic DNA fragment,
gap filling a gap between the transposon DNA and the genomic DNA fragment to form a library of double stranded genomic DNA fragment extension products having primer binding sites at each end within the reaction volume,
amplifying the double stranded genomic DNA fragment extension products to produce amplicons within the reaction volume,
sequencing the amplicons within the reaction volume, and
computationally linking together the amplicons by matching barcodes so as to de novo assemble the genomic DNA.

38. The method of claim 37 wherein the reagent transposome library includes greater than 1,000 transposomes.

39. The method of claim 37 wherein the reagent transposome library includes greater than 10,000 transposomes.

40. The method of claim 37 wherein the reagent transposome library includes greater than 100,000 transposomes.

41. The method of claim 37 wherein the reagent transposome library includes greater than 1,000,000 transposomes.

42. The method of claim 37 wherein the reagent transposome library includes greater than 2,000,000 transposomes.

43. The method of claim 37 wherein the reagent transposome library includes greater than 3,000,000 transposomes.

44. The method of claim 37 wherein the reagent transposome library includes greater than 4,000,000 transposomes.

45. The method of claim 37 wherein the reagent transposome library includes greater than 5,000,000 transposomes.

46. The method of claim 37 wherein the reagent transposome library includes greater than 10,000,000 transposomes.

47. The method of claim 37 wherein the genomic DNA is whole genomic DNA obtained from a single cell.

48. The method of claim 37 wherein the transposase is Tn5 transposase, Mu transposase, Tn7 transposase or IS5 transposase..

49. The method of claim 37 wherein the transposon DNA includes a double-stranded 19 bp Tnp binding site and an overhang, wherein the overhang includes a barcode sequence and a primer binding site at the 5′ end of the overhang.

50. The method of claim 37 wherein bound transposases are removed from the double stranded fragments before gap filling and extending of the double stranded genomic DNA fragments.

51. The method of claim 37 wherein the transposases are Tn5 transposases each complexed with a transposon DNA, wherein the transposon DNA includes a double-stranded 19 bp Tnp binding site and an overhang, wherein the overhang includes a barcode sequence and a primer binding site.

52. The method of claim 37 wherein the genomic DNA is from a prenatal cell.

53. The method of claim 37 wherein the genomic DNA is from a cancer cell.

54. The method of claim 37 wherein the genomic DNA is from a circulating tumor cell.

55. The method of claim 37 wherein the genomic DNA is from a single prenatal cell.

56. The method of claim 37 wherein the genomic DNA is from a single cancer cell.

57. The method of claim 37 wherein the genomic DNA is from a single circulating tumor cell.

58. The method of claim 37 wherein the primer binding site is a specific PCR primer binding site.

59. A method of de novo genomic DNA assembly comprising
contacting transposases with a plurality of transposon DNA within physically separated reaction chambers to form transposomes within each physically separated reaction chamber, wherein each transposon DNA includes a common transposase binding site, a common primer binding site and a barcode sequence, wherein the barcode sequence is the same for all transposon DNA within the same reaction chamber, but different from transposon DNA within other reaction chambers,
collecting the transposomes from each reaction chamber and mixing all the transposomes to form a transposome library
forming a reagent transposome library within a reaction volume wherein substantially all or all transposomes within the reagent transposome library have a unique associated barcode sequence,
contacting genomic DNA with the reagent transposome library within the reaction volume wherein the transposomes bind to target locations along the genomic DNA and the transposase cleaves the genomic DNA into a plurality of double stranded genomic DNA fragments representing a genomic DNA fragment library, with each double stranded genomic DNA fragment including one member of a unique barcode sequence pair on each end of the genomic DNA fragment,
gap filling a gap between the transposon DNA and the genomic DNA fragment to form a library of double stranded genomic DNA fragment extension products having primer binding sites at each end within the reaction volume,
amplifying the double stranded genomic DNA fragment extension products to produce amplicons within the reaction volume,
sequencing the amplicons within the reaction volume, and
computationally linking together the amplicons by matching barcodes so as to de novo assemble the genomic DNA.

60. The method of claim 59 wherein the reaction chambers are tubes, multi-well plates, micro-array chips, micro-wells, micro-reactors, micro-droplets, micro-particles hydrogel or other compartmentalization methods.

61. The method of claim 23 wherein the haplotype-resolved de novo assembly is on a human leukocyte antigen region, V(D)J recombination region or other regions of human single cells.
